(19) United States
(12) Patent Application Publication
(10) Pub. No.: US 2023/0220451 A1
(43) Pub. Date: Jul. 13, 2023

(54) ANALYTE DETECTION METHOD

(71) Applicant: IMPERIAL COLLEGE INNOVATIONS LIMITED, London (GB)

(72) Inventors: Aleksandar Petrov IVANOV, London (GB); Ren REN, London (GB); Joshua Benno EDEL, London (GB)

(21) Appl. No.: 17/927,491

(22) PCT Filed: May 27, 2021
(86) PCT No.: PCT/EP2021/064271

§ 371 Date: Nov. 23, 2022

(30) Foreign Application Priority Data
May 27, 2020 (GB) 2007911.7

(51) Int. Cl. C12Q 1/6825; G01N 33/74; G01N 33/553; G01N 27/447

(52) U.S. Cl. CPC C12Q 1/6825; G01N 33/74; G01N 33/553; G01N 27/44791; C12Q 2600/178; C12Q 1/6886

(57) ABSTRACT

The present invention provides a method of detecting one or more analytes in a target sample, the method comprising: a. providing a nanoparticle dimer adapted to bind the analyte; b. causing the dimer to pass through a nanopore by voltage-driven translocation; c. observing changes in the translocation current; and d. comparing the translocation current profile of the target sample to the translocation current profile of a control sample; wherein a change in the translocation current profile of the target sample versus the control sample indicates the presence of the analyte in the target sample. Also provided is a method of detecting one or more analytes in a target sample, the method comprising: a. providing a nanoparticle adapted to bind the analyte; b. providing a carrier nucleic acid molecule with at least one single-stranded region; c. contacting the carrier nucleic acid molecule and nanoparticle with the target sample, forming a carrier nucleic acid/analyte/nanoparticle complex; b. causing the carrier nucleic acid/analyte/nanoparticle complex to pass through a biological nanopore by voltage-driven translocation; c. observing changes in the translocation current; and d. comparing the translocation current profile of the target sample to the translocation current profile of a control sample; wherein a change in the translocation current profile of the target sample versus the control sample indicates the presence of the analyte in the target sample.

### FIELD OF INVENTION

The present invention provides a method for the detection of one or more analytes in a sample. Specifically, the invention provides a method of analyte detection involving the use of gold nanoparticles (AuNP) dimers and their detection by voltage-driven nanopore translocation.

### BACKGROUND

Early and rapid diagnosis of disease is an essential step in enhancing effectiveness of treatments and in reducing morbidity and mortality. As a non-invasive and cost-effective tool for early diagnosis and prognosis of acute and chronic diseases, the detection of biomarkers, especially at a single-molecule level, have attracted researchers' attention for decades.

Meanwhile, the exquisite design of micro/nano environmental sensing methods and devices allows the detection of analytes at a single molecule level. With size-controllable features, nanopores^{1, 2, 3, 4, 5} have been proven to have the capability of detecting single DNA^{2, 6, 7, 8, 9}, protein^{10, 11, 12}, nanoparticles^{13, 14, 15, 16, 17} and other molecules^{18, 19, 20}. The detection method is straightforward; Targeting molecules are translocated through a nanometer-sized pore using an applied electric field, and the translocations are detected by the transient changes of ionic current. The magnitude and dwell time of the current change provides information on the structural features of the analytes. However, the screening of small biomolecules using nanopore remained remarkably challenging due to their small size, heterogeneous charge, fast translocation and low signal-to-noise ratio.

Attempts have been made to address some of these limitations by functionalizing the nanopore surface with hydrophobic, and positively or negatively charged binding sites^{21, 22, 23}. In addition, aptamer encoded glass nanopores^{24, 25}, chemically modified nanopores with binding sites^{23, 26, 27}, and nanopipette based field-effect-transistors^{28, 29, 30} have also been investigated. However, functionalizing nanopores is often challenging due to complicated fabrication processes and the requirement for careful optimization. As another potential solution, high bandwidth amplifiers have been used to detect small molecules^{31, 32}. Unfortunately, these high-resolution instruments are not capable of differentiating multiple kinds of biomolecules. Therefore, fundamental challenges remain in the sensitive and selective detection of biomarkers such as micro RNA, proteins and small antigen molecules using label-free solid state nanopores.

An alternative solution involves the use of molecular carriers, which include specific sites that interact with the molecules they transport. Singer et al. pioneered the concept of utilising the DNA as molecular carriers³³. Bell et al. used nanopipettes and a carrier based on a 7.2 kbp DNA including a sequence of structural features (dumbbells) as a ‘barcode’ identifying the DNA and a binding site which can interact with targeting antibodies selectively³⁴. Recently, Sze et al. designed a delicate multiple proteins carrier based on λ-DNA with ssDNA overhang which can anchor aptamers to bind targeting proteins (up to three) selectively^{35 and 35a}. Combining the above concepts, more sophisticated molecular carriers have been reported^{36, 37}. However, these designs are restricted by many factors such as the relatively high concentration of targeting analytes, especially proteins, and high ionic strength of buffer/sample required to prevent non-specific binding due to the shielding effect provided by salt ions. Moreover, the folding of long-chain DNA based carrier backbone³⁸ can cause interference in the identification of the analyte signal, while the short carriers are hard to fabricate, purify and embed the binding sites. These approaches are also not capable of sensing small molecules (<15 kDa) due to the poor signal-to-noise ratios.

### SUMMARY OF INVENTION

To address the limitations of the prior art, the present invention provides sensing strategies which aim to increase the sensitivity and selectivity of nanopore biosensing by increasing the size of the analyte, in order to improve the signal-to-noise ratio.

Accordingly, in a first aspect there is provided a method of detecting one or more analytes in a target sample, the method comprising:
a. providing a nanoparticle dimer adapted to bind the analyte;
b. causing the dimer to pass through a nanopore by voltage-driven translocation;
c. observing changes in the translocation current; and
d. comparing the translocation current profile of the target sample to the translocation current profile of a control sample;
wherein a change in the translocation current profile of the target sample versus the control sample indicates the presence of the analyte in the target sample.

The nanoparticle dimer may be adapted to bind the analyte in a number of different ways.

Preferably the dimer comprises two nanoparticles linked by one or more nucleic acids, wherein at least one of the nucleic acids includes an aptamer specific for the analyte. More preferably, the nanoparticles may be linked by partially complementary nucleic acids.

Preferably, the dimer comprises two nanoparticles, each of which is attached to a nucleic acid, and each of the nucleic acids includes part of an aptamer specific for the analyte, such that in the presence of the analyte an aptamer is formed and thereby a dimer is formed.

Preferably, the analyte is a protein.

Preferably, the dimer comprises two nanoparticles, each of which is modified with a single stranded DNA (ssDNA), wherein one ssDNA includes a sequence which is complementary to the sequence of one end of the analyte, and the other ssDNA includes a sequence which is complementary to the sequence of the other end of the analyte, such that in the presence of the analyte a dimer is formed. The analyte may be a nucleic acid, such as DNA or RNA, and may be single-stranded or double-stranded with a single-stranded overhang. The analyte is preferably miRNA, but may be any kind of RNA such as mRNA, tRNA, siRNA, gRNA, ncRNA, exRNA, crRNA, or lncRNA. Preferably, the dimer comprises two nanoparticles, each of which is conjugated to an antibody specific to a different epitope on the analyte, such that in the presence of the analyte a dimer is formed. Preferably, the analyte is an antigen.

Preferably, the nanoparticles in the dimer are of substantially the same diameter. Alternatively, the nanoparticles in the dimer may be of different diameters.

Preferably, the nanopore is the tip of a nanopipette.

Preferably, the nanoparticles are gold nanoparticles (AuNPs).

Preferably, the target sample is a biological sample selected from blood, serum, lymph, sputum, urine, faeces, semen, sweat, tears, amniotic fluid, cerebrospinal fluid (CSF) and wound exudate.

The biological sample may alternatively be any other bodily fluid or secretion in a state of health or disease.

The present inventors have exemplified the first aspect of the invention using three strategies. The Examples of the present application relate to gold nanoparticles (AuNPs), but it will be appreciated that the invention is applicable to any nanoparticle which is suitable (in terms of its size and surface charge) for electrical detection when passed through a nanopore using voltage-driven translocation.

The first strategy—for transporting and identifying relatively bigger single proteins (>35 kDa)—is implemented by a carrier which is a dumbbell system comprising two nanoparticles with an aptamer located at the middle of a DNA linker (FIG. 1a). Each end of the linker is attached to one of the nanoparticles. Aptamers are ssDNA or RNA oligonucleotides that are capable of binding target molecules with high specificity and affinity, where the sequence can be selected by SELEX (systematic evolution of ligands by exponential enrichment). The specific aptamer modified dimer carriers thus bind to target proteins selectively. Then the sub-structure of protein/carrier complexes can be identified when they pass through the nanopore along with high-resolution translocation signals. A specific example of such a configuration using AuNPs and ssDNA linkers is shown in FIG. 6.

The second strategy—for detecting tiny antigen molecules (<15 kDa)—is achieved by a pair of monomer (nanoparticle) probes, one being conjugated to an antibody to a target antigen and the other being conjugated to a complementary secondary antibody (FIG. 1b). The monomer (nanoparticle) probes will self-assemble to dimer molecules by the linkage of the antibody-antigen-antibody sandwich bridge after the addition of the specific antigen. In this embodiment, the method of the first aspect of the invention can also be seen to include a step of causing the nanoparticles to form a dimer.

The two strategies can be combined (FIG. 1d): one nanoparticle is functionalised with ssDNA with a part of aptamer sequence at the end while the other nanoparticle is functionalised with ssDNA that includes the rest of aptamer sequence; when the target protein is present, the two aptamer parts will link together to form an aptamer which binds to the protein, leading to the conjugation of the nanoparticle monomers. It can thus be seen that in this embodiment the dimer is formed by formation of the aptamer. In this embodiment, the method of the first aspect of the invention can also be seen to include a step of causing the nanoparticles to form a dimer.

A third strategy can be used for the selective detection of nucleic acid molecules such as miRNA (FIG. 1c). Two populations of nanoparticles can be modified to each contain half (or at least two different portions) of a sequence complementary to an miRNA sequence. In the presence of miRNA, the monomeric nanoparticles will self-assemble and dimerize.

In all strategies, the translocation signal of the nanoparticle dimer compared to individual nanoparticles indicates the presence of the analyte. This allows for very accurate detection of analytes based on the peak shape. All strategies can be adapted to different targets (including small molecules such as small peptides, proteins and nucleic acids such as miRNAs) by changing the aptamer sequence or antibody to the required target. In addition, these strategies can be adapted to quantify concentration of the target. The strategies also allow detection of analytes in complex media as the analytes are not detected directly but rather via the formation of dimers.

The present inventors have also devised a strategy for using nanopore monomers for sensing analytes using functionalised nanoparticle probes and DNA carriers by biological nanopores. These aspects of the invention are shown in FIGS. 13 and 14, which illustrate the methods for AuNP probes.

In a second aspect, the present invention provides a method of detecting one or more analytes in a target sample, the method comprising:
a. providing a nanoparticle adapted to bind the analyte;
b. providing a carrier nucleic acid molecule with at least one single-stranded region;
c. contacting the carrier nucleic acid molecule and nanoparticle with the target sample, forming a carrier nucleic acid/analyte/nanoparticle complex;
b. causing the carrier nucleic acid/analyte/nanoparticle complex to pass through a biological nanopore by voltage-driven translocation;
c. observing changes in the translocation current; and
d. comparing the translocation current profile of the target sample to the translocation current profile of a control sample;
wherein a change in the translocation current profile of the target sample versus the control sample indicates the presence of the analyte in the target sample.

The nanoparticle dimer may be adapted to bind the analyte in a number of different ways.

Preferably, the carrier nucleic acid molecule includes an aptamer specific for the analyte and the nanoparticle is conjugated to an antibody specific to a different epitope on the analyte from that to which the aptamer binds. Preferably, the analyte is a protein.

Preferably, the carrier nucleic acid molecule is a single stranded DNA (ssDNA) which includes a sequence that is complementary to the sequence of one end of the analyte and the nanoparticle is modified with a ssDNA which includes a sequence that is complementary to the sequence of the other end of the analyte. The analyte may be a nucleic acid, such as DNA or RNA, and may be single-stranded or double-stranded with a single-stranded overhang. The analyte is preferably miRNA, but may be any kind of RNA such as mRNA, tRNA, siRNA, gRNA, ncRNA, exRNA, crRNA, or lncRNA.

Preferably, the biological nanopore is alpha-hemolysin.

Preferably, the nanoparticle is a gold nanoparticle (AuNP).

Preferably, the target sample is a biological sample selected from blood, serum, lymph, sputum, urine, faeces, semen, sweat, tears, amniotic fluid, cerebrospinal fluid (CSF) and wound exudate.

The biological sample may alternatively be any other bodily fluid or secretion in a state of health or disease.

### DESCRIPTION OF DRAWINGS

FIG. 1. Schematic of dimer protein carrier and monomer antigen probes.

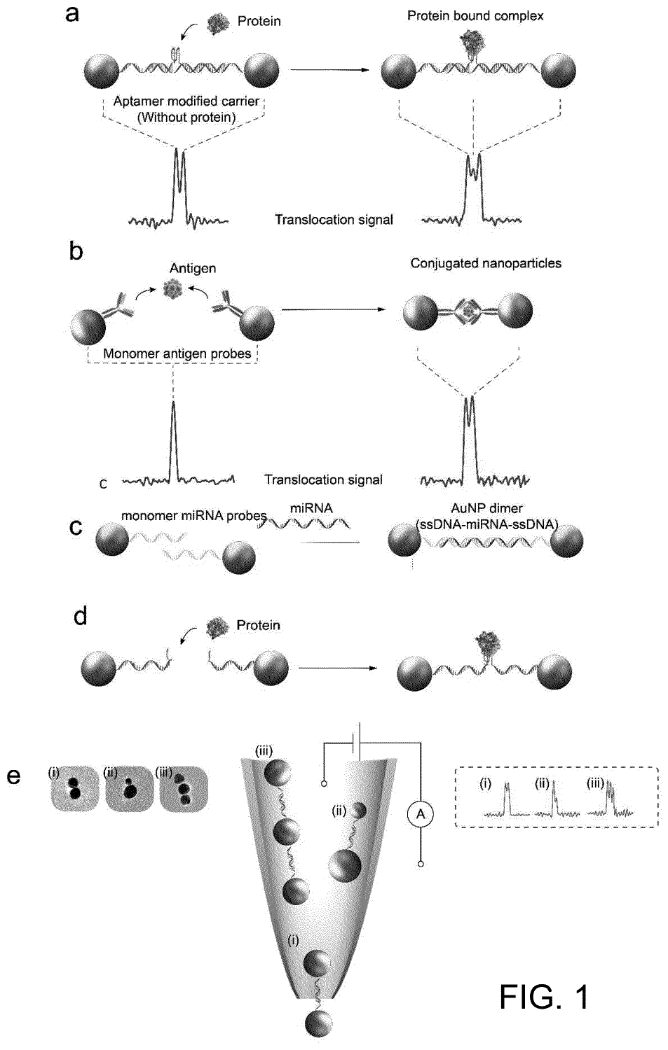

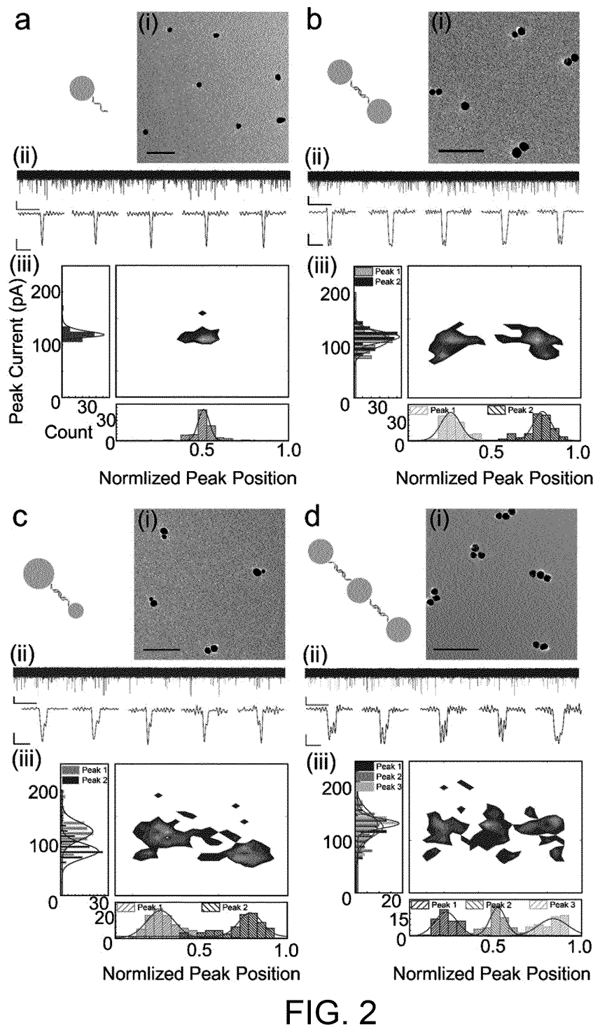

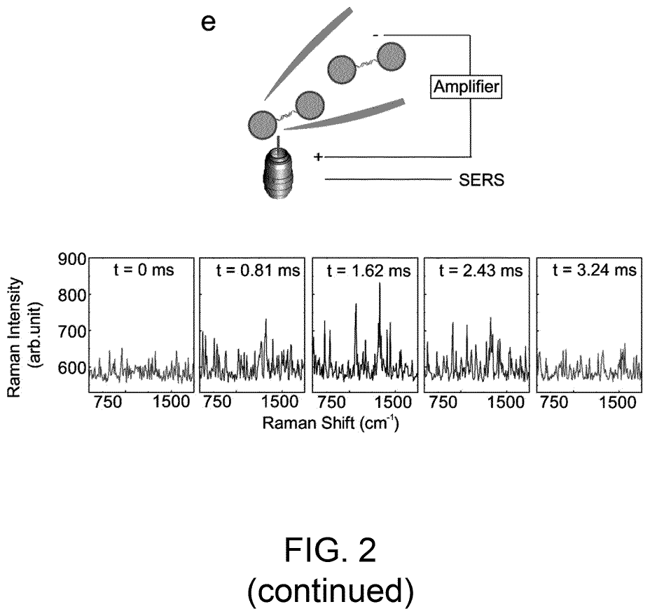

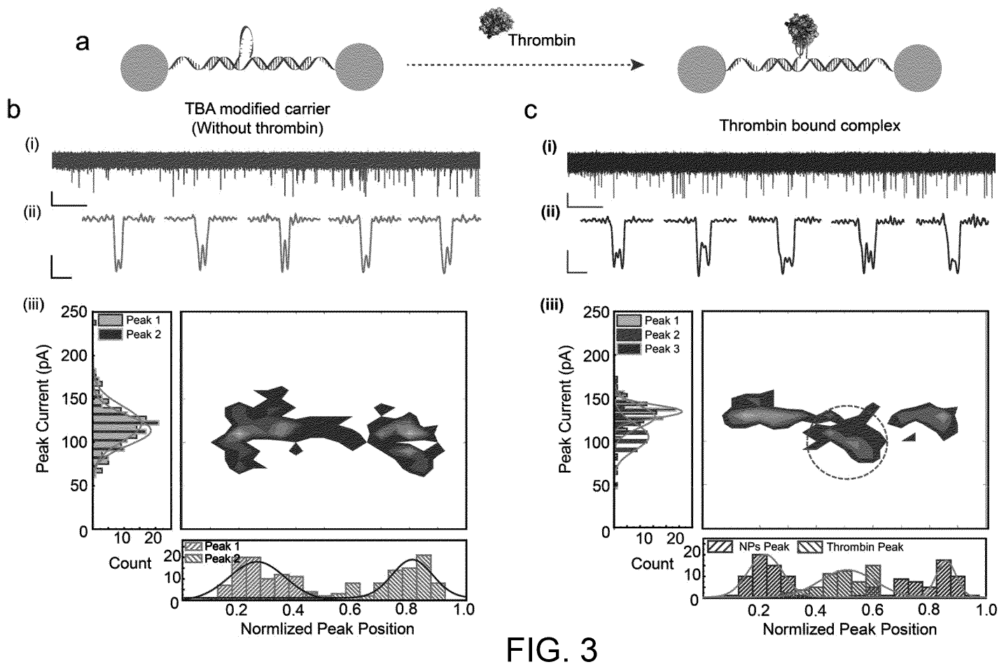

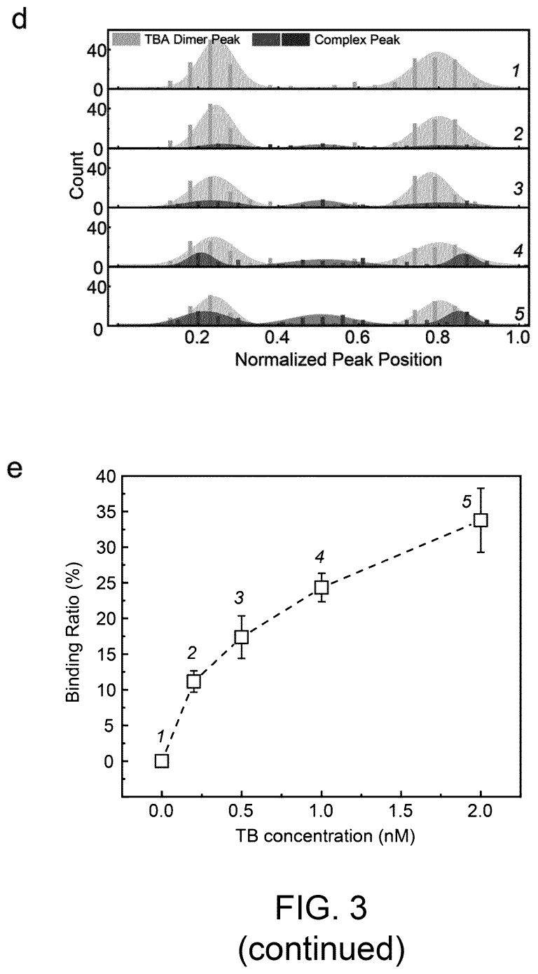

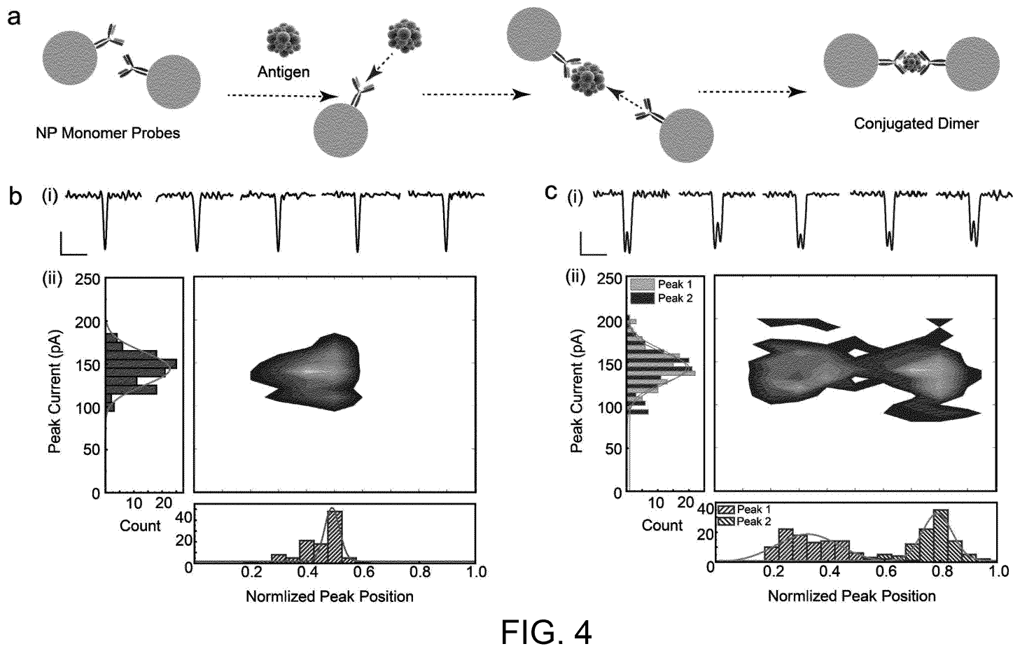

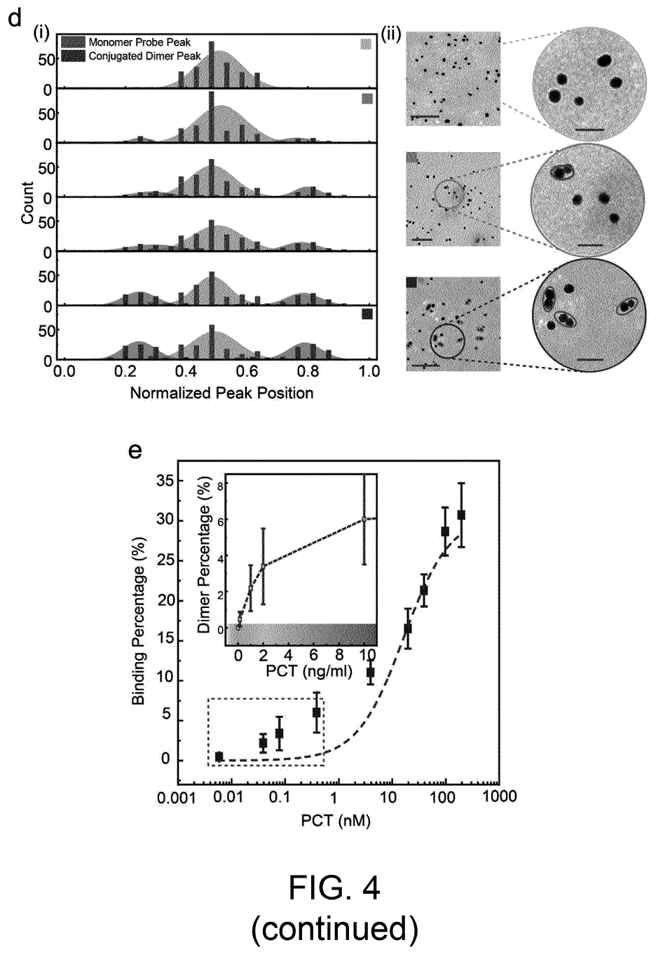

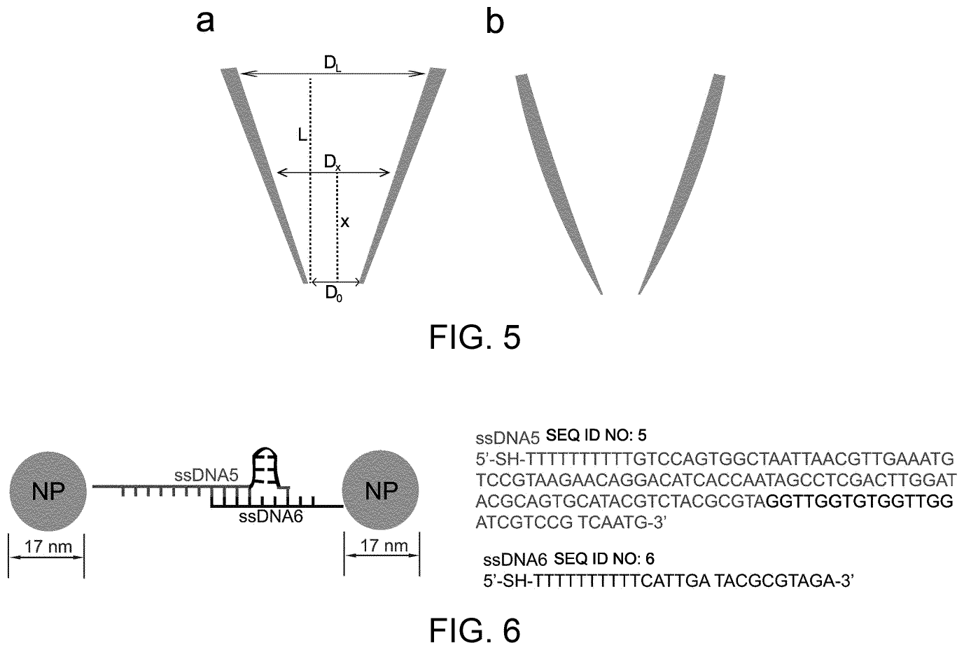

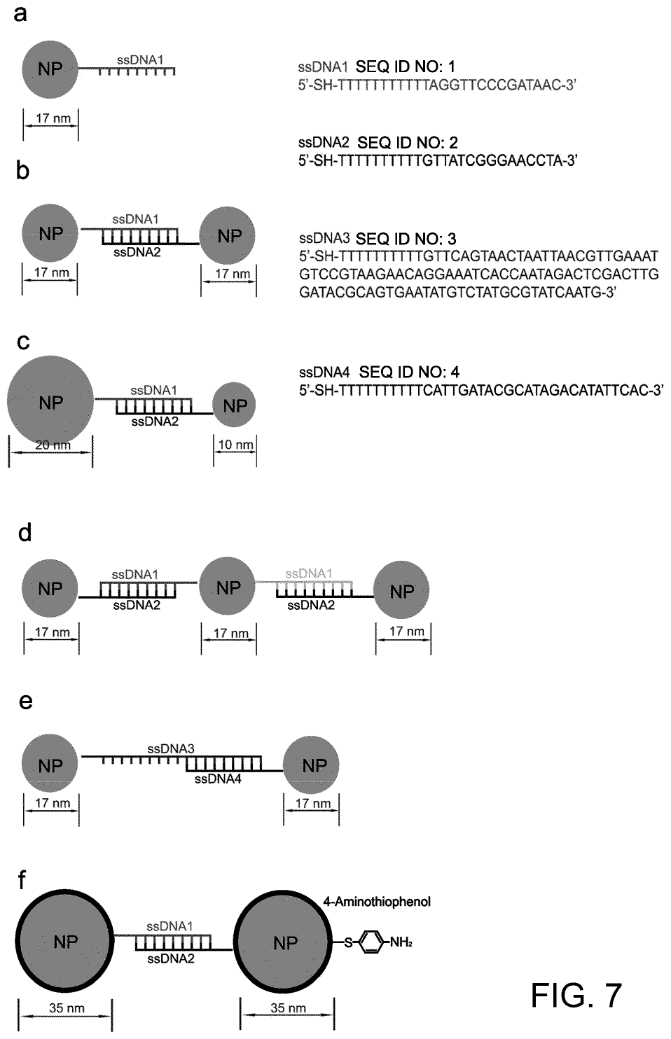

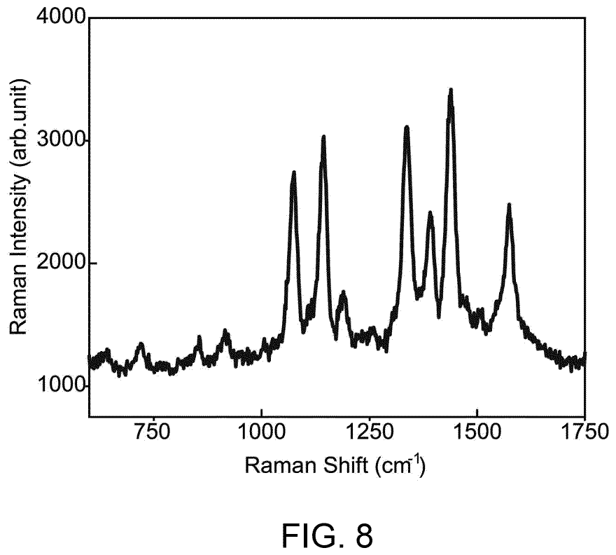

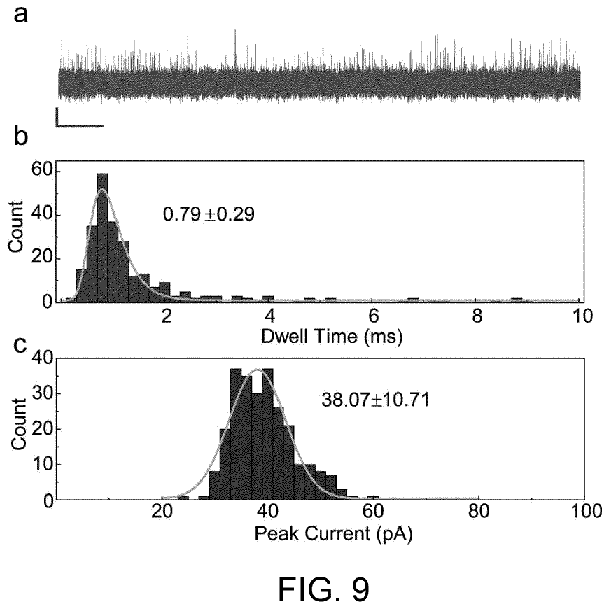

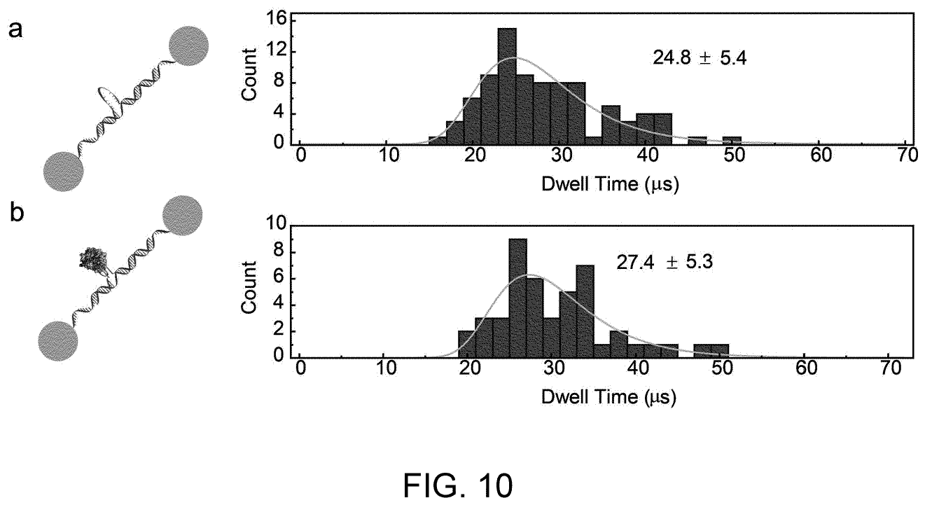

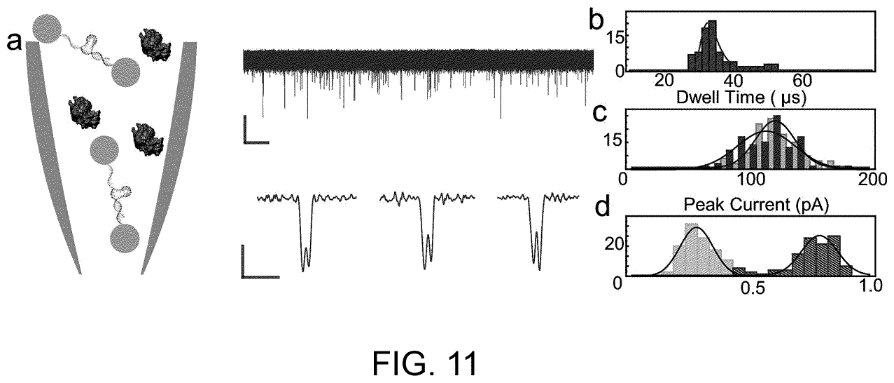

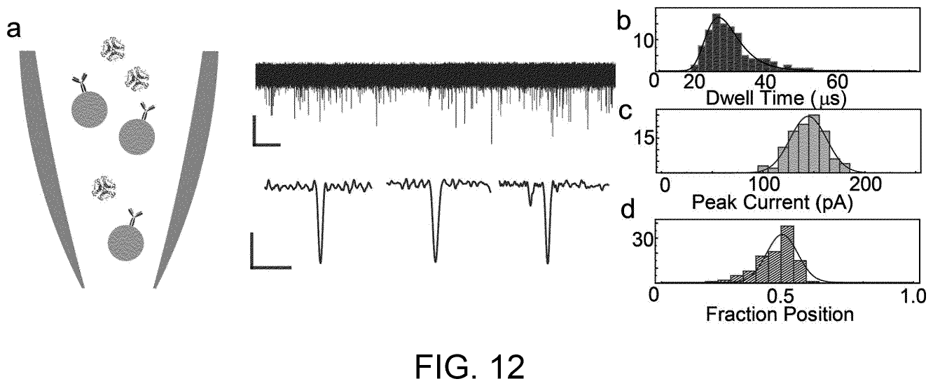

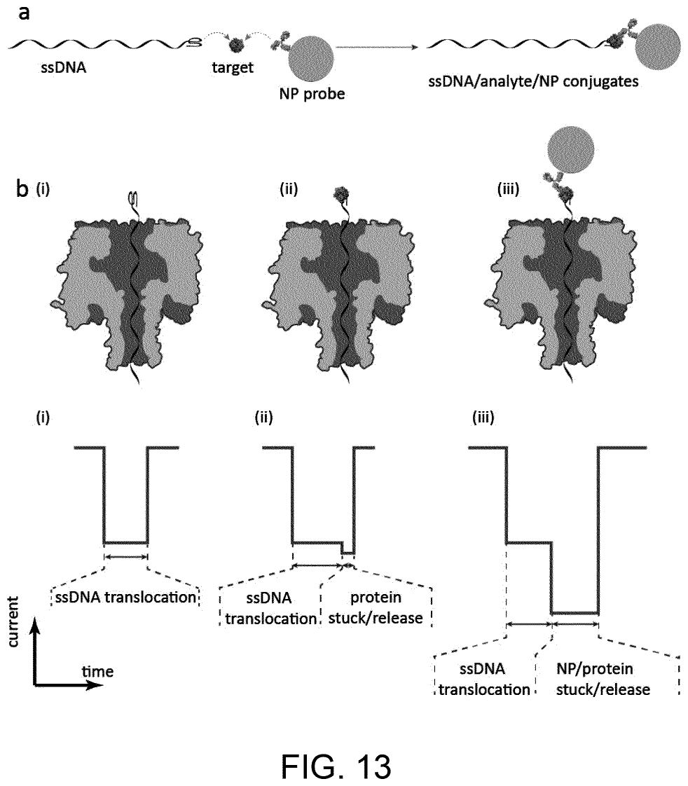

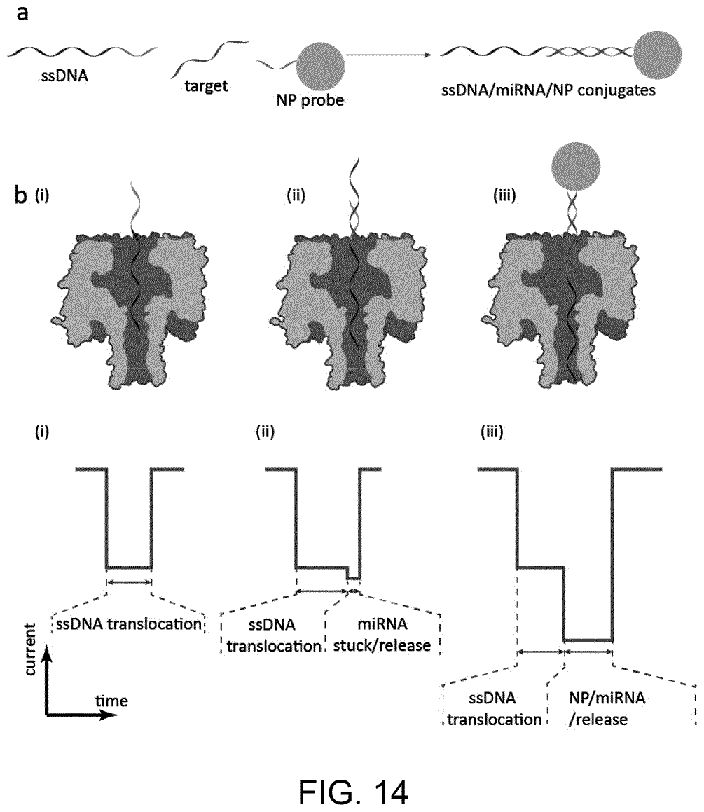

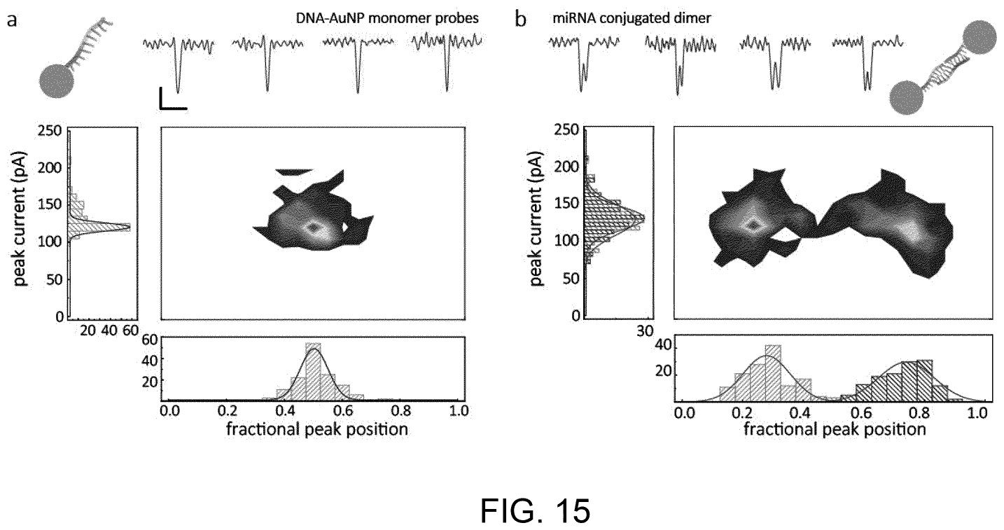

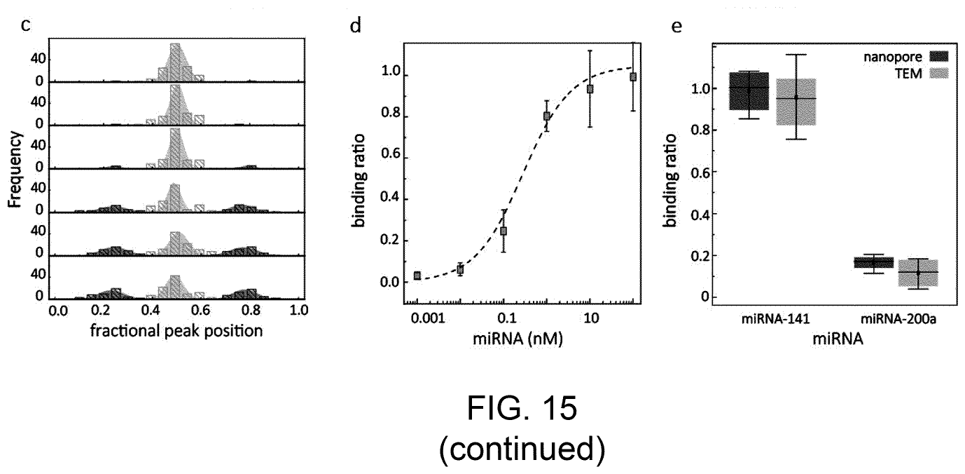

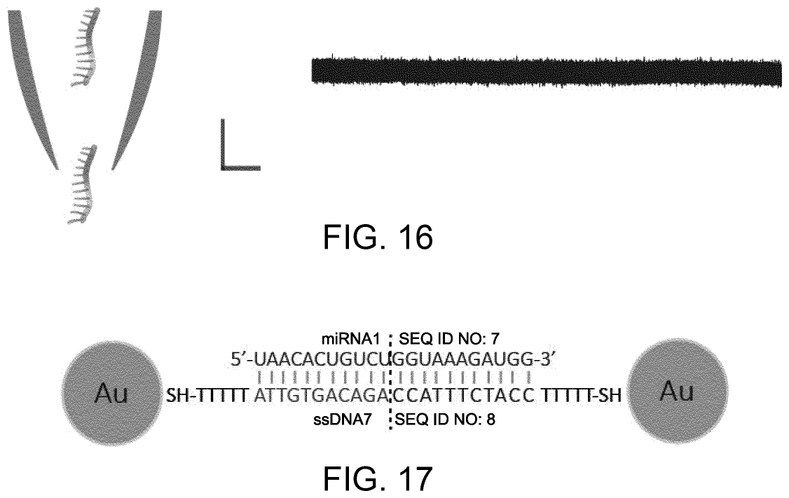

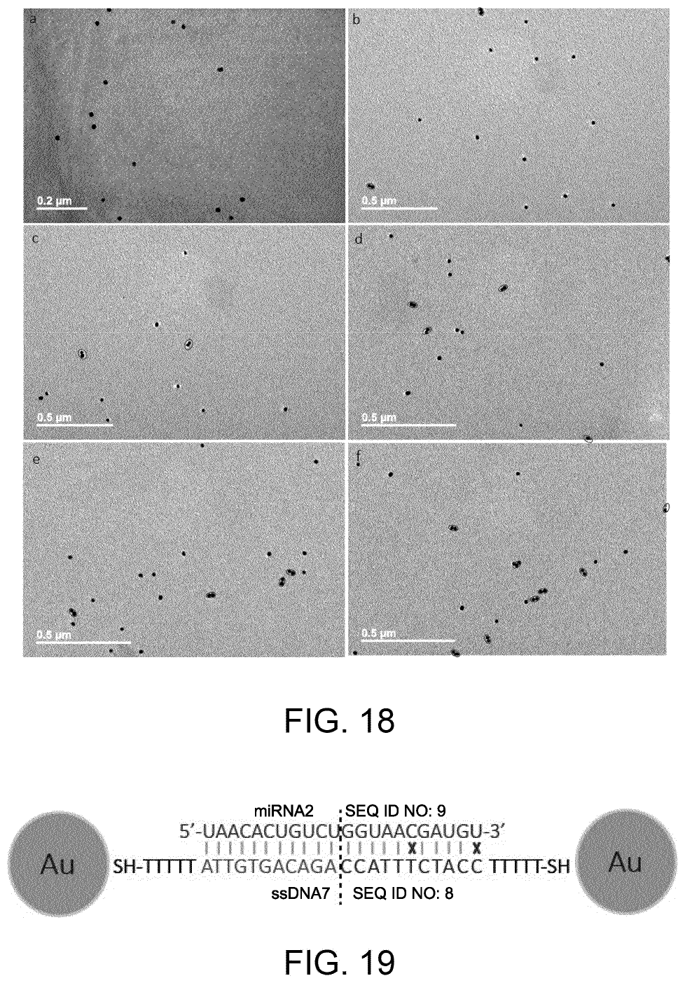

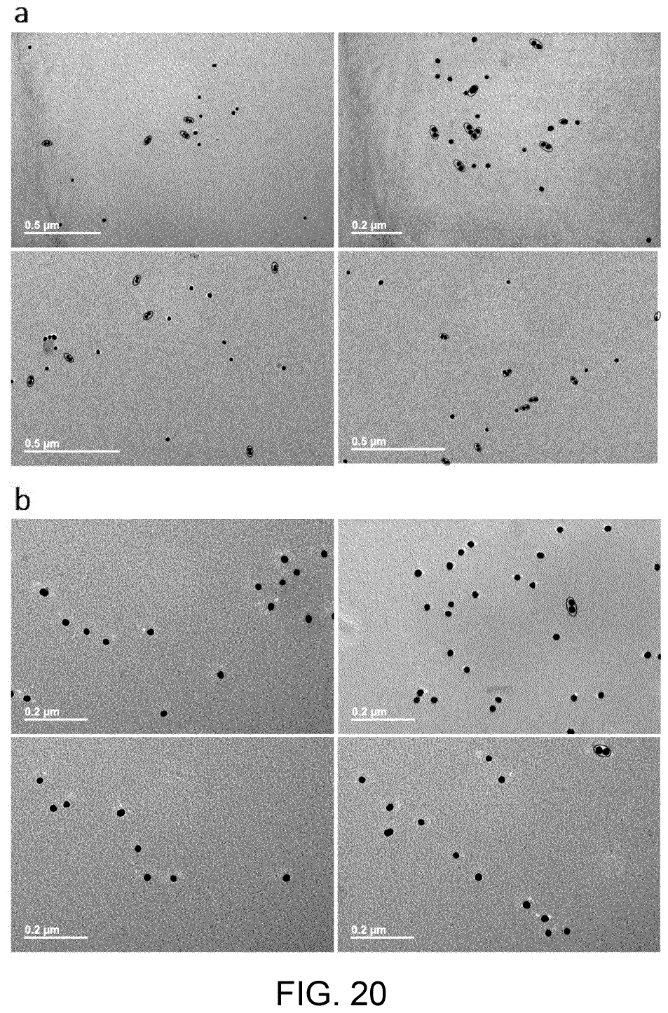

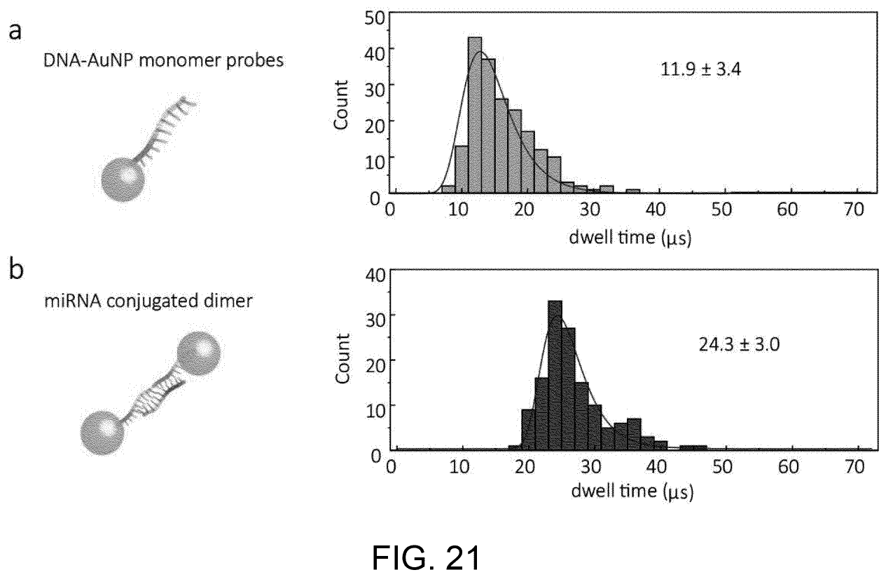

The dimer carrier (a) is based on a dumbbell system where two nanoparticles (for example Au nanoparticles (17-20 nm)) are linked by a ^{˜}110-mer DNA bridge which is engineered to contain an aptamer sequence that binds to target protein molecules. The monomer probes (b) contain two types of nanoparticle, where each nanoparticle is modified with a different antibody, and the two nanoparticles bind together with the presence of the target antigen. Driven by the electrical field, the nanoparticle dimer carriers with target protein or the dimers with the antibody-antigen-antibody sandwich bridge translocate from inside nanopipette to outside. (c) A similar strategy can be used for the selective detection of miRNA. Two populations of NPs can be modified to each contain half of the complementary miRNA sequence. In the presence of miRNA the monomeric NPs will self-assemble and dimerize. (d) The two strategies shown in parts (a) and (b) can be combined: one nanoparticle is functionalised with ssDNA with a part of aptamer sequence at the end while the other nanoparticle is functionalised with ssDNA that includes the rest of aptamer sequence; when the target protein is present, the two aptamer parts will link together and bind to the protein, leading to the conjugation of the nanoparticle monomers. The changes of translocation events represent the sensing of target protein or antigens at single molecule level. Also, by counting the percentage of these signatures, the concentration of the specific target molecules can be achieved. (e) The schematic of screening the substructure of the nanoparticle conjugates by using a nanopipette.

FIG. 2. The TEM images, current-time trace, individual events, single molecule SERS and statistics of a series of AuNP based nanostructures.

The TEM (Scale bar, 100 nm), current-time trace (Scale bar: vertical 50 pA, horizontal 5 s), individual events (Scale bar: vertical 50 pA, horizontal 20 μs) and statistics for a AuNP monomer b AuNPs symmetrical dimer c AuNPs asymmetrical dimer and d AuNPs trimer. TEM images (i) indicate the clear geometry of the molecules. All the translocation experiments were performed in 50 mM KCl, 10 mM Tris-HCl, 1 mM EDTA and at a potential of −600 mV. Each current-time trace (ii) is recorded with 1 is sampling rate and filtered using a low-pass Bessel filter with a cut-off frequency of 100 KHz. Typical individual translocation events are also shown in (ii) as they can be distinguished easily from others. Surface plots along with peak current and normalized peak position histograms are shown in (iii) clearly indicate that the translocation signal is capable of simulating the nanostructure where the size/charge of each molecule and even the distance between them. e PEG modified 4-ATP loaded 35 nm AuNP dimer which is capable of enhancing the Raman signal of the Raman dye in the nanogap. f Schematic of single molecule SERS experimental arrangement. The dimers were loaded into the gold coated nanopipette and a negative bias voltage was applied to drive the molecules to pass through the nanopore. The electrical signal and Raman signal can be recorded simultaneously. g Raman spectra showing the translocation of dimers. An example of 4.05 ms translocation using a −800 mV potential at an 810 is acquisition time. These spectra show single AuNP dimer is translocating through the nanopore over the time period of 4.05 ms.

FIG. 3. Nanopore sensing of thrombin with AuNP dimer thrombin carriers.

a The schematic of the dimer carriers, which engineered to contain aptamer sequences, deliver and sense the target protein. b-c The representative current-time trace (Scale bar: vertical 50 pA, horizontal 5 s), individual translocation events (Scale bar: vertical 50 pA, horizontal 20 μs) and statistics for AuNPs dimer thrombin carriers and AuNPs dimer with thrombin complex. The translocation experiments were performed in 50 mM KCl, 10 mM Tris-HCl, 1 mM EDTA and at a potential of −600 mV. Each trace is recorded with 1 is sampling rate and filtered using a low-pass Bessel filter with a cut-off frequency of 100 KHz. d Under different concentration of thrombin (From top to bottom, 0 nM, 0.2 nM, 0.5 nM, 1 nM, and 2 nM), the normalized peak position of the dimer carriers or the carrier and target protein complexes. e Binding assay for a 1 nM dimer carrier concentration incubated with increasing thrombin concentration ranging from 0 to 2 nM. Error bars represent the standard deviation of three independent experimental repeats with different nanopipettes.

FIG. 4. Nanopore sensing of procalcitonin (PCT) via a pair of AuNP monomer probes.

a The schematic of two AuNP monomer probes with different antibody will self-assemble to dimer with the presence of antigen (procalcitonin (PCT)). b-c The individual translocation events (Scale bar: vertical 50 pA, horizontal 20 μs) and statistics of AuNPs monomer probes conjugated dimers which linked by antibody-antigen-antibody sandwich bridges. All the translocation experiments were performed in 50 mM KCl, 10 mM Tris-HCl, 1 mM EDTA and at a potential of −600 mV. The data was recorded with 1 is sampling rate and filtered using a low-pass Bessel filter with a cut-off frequency of 100 KHz. d The normalized peak position of the monomer probe and conjugated dimer with different concentration of PCT (From top to bottom, 0 nM, 80 pM, 20 nM, 40 nM, 100 nM and 200 nM). The TEM images are used to confirm the result of binding ratio (Scale bar: 200 nm, enlarged, 50 nm). e the binding curve of 1 nM AuNP monomer PCT probes incubating with PCT ranging from 0 to 400 nM. The inset is the sensing of PCT in clinical diagnostic range. Error bars of binding ratio analysis represent the standard deviation of three independent experimental repeats.

FIG. 5. Schematic representation of a nanopipette.

a the model of the conical nanopore for effective length calculation. b the reality nanopore shape model.

FIG. 6. The details of AuNP dimer thrombin carrier.

The AuNP dimer thrombin carrier is self-assembled by an AuNP with a 115-mer ssDNA (ssDNA5) containing 15-mer thrombin-binding aptamer (TBA) sequence (shown in black in FIG. 6) and an AuNP with the complementary part to the sequence aside of the aptamer part (ssDNA6). After the self-assembling, the protein carrier with a prominence of aptamer can sense the thrombin selectively. It is noteworthy that there are 8 random bases close to the aptamer sequence to minimize the steric hindrance.

FIG. 7. The details of AuNP based nanostructures.

a AuNP monomer. One 17 nm AuNP modified by a ssDNA (ssDNA1). b AuNPs symmetrical dimer. Two 17 nm AuNPs linked by a DNA bridge (35 bases long) (formed by hybridisation of ssDNA1 and ssDNA2). c AuNPs asymmetrical dimer. One 20 nm AuNP and one 10 nm AuNP linked by a DNA bridge (formed by hybridisation of ssDNA1 and ssDNA2) (35 bases long). d AuNPs trimer. Three AuNPs linked by two DNA bridges (each formed by hybridisation of ssDNA1 and ssDNA2) (each 35 bases long). e AuNPs symmetrical dimer with long linker. Two 17 nm AuNPs linked by a DNA bridge (formed by hybridisation of ssDNA3 and ssDNA4) (117 bases long). f The AuNP symmetrical dimers with Raman reporter. Two 4-aminothiophenol (4-ATP) modified (shown as black outline) 35 nm AuNPs linked by a DNA bridge (formed by hybridisation of ssDNA1 and ssDNA2) (35 bases long).

FIG. 8. The SERS spectrum of the 4-ATP modified AuNP symmetrical dimer in bulk solution.

SERS spectrum of 4-ATP dye present in nanogap of AuNP symmetrical dimer. Spectrum of 4-ATP dye shows extremely high enhancement of the 1138, 1387, and 1571 cm⁻¹ vibrational bands of 4-ATP.

FIG. 9. Translocation of PEG stabilized 35 nm AuNP Dimer functionalized with 4-ATP dye.

The current-time trace (Scale bar: vertical 50 pA, horizontal 5 s and statistics for 1 nM Raman sample (35 nm AuNP Dimer functionalized with 4-ATP dye, protected by SH-PEG). All the translocation experiments were performed in 100 nM KCl, 10 mM Tris-EDTA. The current-time trace is recorded with 10 us sampling rate and filtered using a low-pass Bessel filter with a cut-off frequency of 10 KHz. The dwell time of this sample is much longer than others in this work because the PEG modified molecules has high viscidity. Moreover, the charge of the particles is shield so they move slowly and some unpredictable rotation may happen when the electric field applied.

FIG. 10. The translocation dwell time of AuNP dimer carrier with and without thrombin.

The dwell time of the translocation of a AuNP dimer thrombin carrier b AuNP dimer carrier with thrombin complex.

FIG. 11. The control experiment of AuNP dimer thrombin carrier

The current-time trace (Scale bar: vertical 50 pA, horizontal 5 s), individual events (Scale bar: vertical 50 pA, horizontal 20 μs) and statistics for 1 nM AuNP dimer thrombin carrier incubated with 1 nM lysozyme. All the translocation experiments were performed in 50 mM KCl, 10 mM Tris-EDTA. The current-time trace is recorded with 1 μs sampling rate and filtered using a low-pass Bessel filter with a cut-off frequency of 100 KHz. There are no triple peak events but double peak events, corresponding to unbound thrombin carriers.

FIG. 12. The control experiment of AuNP monomer PCT probe

The current-time trace (Scale bar: vertical 50 pA, horizontal 5 s), individual events (Scale bar: vertical 50 pA, horizontal 20 μs) and statistics for 1 nM AuNP monomer PCT probes incubated with 1 nM Insulin. All the translocation experiments were performed in 50 mM KCl, 10 mM Tris-EDTA. The current-time trace is recorded with 1 is sampling rate and filtered using a low-pass Bessel filter with a cut-off frequency of 100 KHz. There are no double peak events but single peak events, corresponding to free PCT probes.

FIG. 13 Sensing analytes using antibody-functionalised NP probes and DNA carriers by biological nanopores.

a With the presence of the target protein, the aptamer-modified ssDNA carrier and the NP antibody probe will self-assemble to a ssDNA-protein-NP sandwich complex. b Schematic of working principle for nanopore sensing using DNA-NP self-assembly (top: working diagrams, bottom: typical signals). i. When the free ssDNA carrier translocates through the biological nanopore, the ionic current will be blocked by the DNA molecule and show a drop-down. ii. Since the protein cannot enter the nanopore, for ssDNA bound with target protein, the signal will show a subpeak due to the stuck or release of target protein. iii. With the form of ssDNA-protein-NP complex, the subpeak will be amplified due to the existence of NP. NP serves as a signal amplifier and effectively increases signal-to-noise ratio.

FIG. 14 Sensing miRNAs using DNA-functionalised NP probes and DNA carriers by biological nanopores.

a With the presence of the target miRNA, the ssDNA carrier with a complementary DNA (cDNA1) end to miRNA and the NP probe modified with the rest complementary DNA (cDNA2) will self-assemble to a ssDNA-miRNA-NP complex due to DNA-RNA hybridisation. b Schematic of working principle for nanopore sensing using DNA-NP self-assembly (top: working diagrams, bottom: typical signals). i. When free ssDNA translocates through the biological nanopore, the ionic current will be blocked by the DNA molecule and show a drop-down. ii. Since the hybridised dsDNA cannot enter the nanopore, for ssDNA bound with target miRNA, the signal will show a subpeak due to the stuck or release of target miRNA. iii. With the form of ssDNA-miRNA-NP complex, the subpeak will be amplified due to the existence of NP. NP serves as a signal amplifier and effectively increases signal-to-noise ratio.

FIG. 15 Nanopore sensing of mRNA-141.

(a) AuNP monomer miRNA-141 molecular probes. Representative individual translocation events are shown (Scale bar: vertical 50 pA, horizontal 20 μs) along with associated statistics. (b) Conjugated dimers with miRNA141 linked between 2 NP monomers. All the translocation experiments were performed in 50 mM KCl, 10 mM Tris-EDTA and at a potential of −600 mV. (c) The normalised peak position of the monomer probe and conjugated dimer with different concentration of mRNA-141 (From top to bottom, 0 nM, 10 pM, 100 pM, 1 nM, 10 nM and 100 nM). (d) the binding curve of 2 nM AuNP monomer miRNA-141 probes incubating with the target miRNA ranging from 0 to 100 nM. (e) The comparison of the detection of miRNA-141 and miRNA-200a using AuNP monomer miRNA-141 probes. Error bars represent the standard deviation of three independent experimental repeats.

FIG. 16. Sensing of miRNA without AuNP probes.

The current-time trace of miRNA-141 (10 nM) nanopore sensing (Scale bar: vertical 50 pA, horizontal 5 s). The translocation experiments were performed in 50 mM KCl, 10 mM Tris-EDTA, and at a potential of −600 mV. The current-time trace is recorded at a 1 MHz sampling rate and filtered using a low-pass Bessel filter with a cut-off frequency of 100 kHz. There are no observable translocations.

FIG. 17. Schematic of miRNA-141 and AuNP monomer probes.

The miRNA-141 detection probes contain two types of ssDNA functionalized AuNP monomers. Each of them was modified by an 11 base recognition chain, which can hybridize with half of 22-base-long miRNA-141 (grey sequence in the figure). With the addition of the target, the monomer probes form a dimer. It is noteworthy that there are 5 T close to the ssDNA recognition sequence to minimize the steric hindrance.

FIG. 18. TEM images of AuNP monomer probes incubated with different levels of miRNA.

The TEM images (scale bars, 200 nm) of AuNP monomer probes incubated with (a) 0 nM (b) 10 pM (c) 100 pM (d) 1 nM (e) 10 nM (f) 100 nM miRNA-141.

FIG. 19. The schematic of a miRNA-141 probe binding to miRNA-200a.

This was used as a control experiment and consisted of a two-base mismatch, which heavily decreases the affinity of hybridization.

FIG. 20. TEM images of the AuNP monomer miRNA-141 probe with the addition of miRNA-141 and miRNA-200a.

A large number of miRNA-141 probes (2 nM) dimerized to dumbbell molecules with the presence of (a) 100 nM miRNA-141, which is fully matched with the probe sequence, where only very few dimer molecules generated with the addition of (b) 100 nM miRNA-200a, which has two base mismatch with the AuNP probe.

FIG. 21. Dwell times for the translocation of (a) DNA-AuNP monomer probes (b) miRNA conjugated dimers.

### DESCRIPTION OF SEQUENCES

The sequences referred to in the Figures are as follows:

| SEQ | Nucleic | | |
|---|---|---|---|
| ID NO | Acid | FIG. | Sequence |
| SEQ ID | ssDNA1 | 7 | 5′-SH-TTTTTTTTTTTAGGTTCCCGATA |
| NO: 1 | | | AC-3′ |
| SEQ ID | ssDNA2 | 7 | 5′-SH-TTTTTTTTTTGTTATCGGGAACC |
| NO: 2 | | | TA-3′ |
| SEQ ID | ssDNA3 | 7 | 5′-SH-TTTTTTTTTTGTTCAGTAACTAA |
| NO: 3 | | | TTAACGTTGAAATGTCCGTAAGAACAGGA |
| | | | AATCACCAATAGACTCGACTTGGATACGC |
| | | | AGTGAATATGTCTATGCGTATCAATG-3′ |
| SEQ ID | ssDNA4 | 7 | 5′-SH-TTTTTTTTTTCATTGATACGCAT |
| NO: 4 | | | AGACATATTCAC-3′ |
| SEQ ID | ssDNA5 | 6 | 5′-SH-TTTTTTTTTTGTCCAGTGGCTAA |
| NO: 5 | | | TTAACGTTGAAATGTCCGTAAGAACAGGA |
| | | | CATCACCAATAGCCTCGACTTGGATACGC |
| | | | AGTGCATACGTCTACGCGTAGGTTGGTGT |
| | | | GGTTGGATCGTCCGTCAATG-3′ |
| SEQ ID | ssDNA6 | 6 | 5′-SH-TTTTTTTTTTCATTGATACGCGT |
| NO: 6 | | | AGA-3′ |
| SEQ ID | miRNA1 | 17 | 5′-UAACACUGUCUGGUAAAGAUGG-3′ |
| NO: 7 | | | |
| SEQ ID | ssDNA7 | 17 and | 5′-SH-TTTTTCCATCTTTACCAGACAGT |
| NO: 8 | | 19 | GTTATTTTT-SH-3′ |
| SEQ ID | miRNA2 | 19 | 5′-UAACACUGUCUGGUAACGAUGU-3′ |
| NO: 9 | | | |

### DETAILED DESCRIPTION

The ability to measure specific, selective biomarker molecules at single molecule level in physicians' surgeries and clinics has the potential to revolutionize disease diagnosis, monitoring, and therapy. Early and rapid diagnosis is an important factor to enhance the effectiveness of treatment. In many methods of early stage diagnosis, traditional biomarker detection methods like PCR and antigen-detecting ELISAs are widely used but limited due to many factors: 1) Low concentration of target biomarker molecules. People may be poor producers of an antibody or may have some interfering substance in their blood. The amount of antibody, consequently, may be too low to measure accurately or may go undetected. 2) Lack of single molecule data. This may cause the unspecific detection or recognise all isoforms of one same protein in a sample. 3) Other factors such as complex sample preparation and time consumption. Therefore, a fast, accurate and specific single molecule detection method is urgently needed.

Nanopores provide a label-free platform for sensing single biomolecules. Under applied potential, charged molecules will pass through the nanoscale pore and the resulting ionic current can be measured with standard electrophysiological techniques. Normally, long strand DNA molecules and large protein molecules can be distinguished easily by recognising the ionic current signal. However, this single molecule method is still challenging due to the low concentration in biological samples, fast translocation time of the analytes, poor analyte selectivity and low signal-to-noise ratio. Especially on the detection of protein molecules with small size and heterogeneous charge, such as lysozyme, thrombin and alpha synuclein, the single molecule signal becomes undetectable as the speed at which these molecules get transported through the nanopore is often too quick and hence hard to resolve. The present invention provides flexible, efficient and low-cost strategies to sense biomolecules of different sizes using a high resolution nanopore system.

The first aspect of the invention provides a series of molecular carriers and probes based on a nanoparticle dimer system, preferably a gold nanoparticle (AuNP) dimer system, which can deliver small analytes through a nanopore with improved signal-to-noise ratios.

The principle underlying the invention is increasing the effective size of the analyte using the nanoparticle dimer system. The signal of the dimer/analyte complex passing through a nanopore via voltage-driven translocation can be distinguished from the unbound dimer, allowing for detection even where the signal of the analyte cannot easily be detected.

The target analyte binds to dumbbell-shaped dimer carriers, with the corresponding aptamer located in the middle part, and then will be transported through a nanopore such as a fine-tuning nanopipette. Recorded by a high-bandwidth instrument, the high-res signal of nanoparticle monomers, nanoparticle dimers and nanoparticles with target protein can be differentiated by analysing the translocation events.

Moreover, this dimer system can be used to detect even smaller molecules which are highly likely undetectable in single molecule level. When the target protein is added into the solution, two nanoparticle monomers with different binding sites (such as antibody 1 and antibody 2) will link to each other and generate a nanoparticle-Antigen-nanoparticle dumbbell molecule because they will both bind to the target molecule. By detecting the ratio between the monomer and dimer, the high-res nanopore system not only can sense the antigens but also can quantify the trace amount concentration of them.

As described herein, the present invention is applicable to any nanoparticle which is suitable (in terms of its size and surface charge) for electrical detection when passed through a nanopore using voltage-driven translocation.

The sensing approaches of the present application are based on the differentiation of nanoparticles or nanoparticle-based conjugates. Herein, all nanoparticles with a suitable size and charge can be used in these methods. This includes nanoparticles formed of metals other than gold, alloys, polymers and silica. With different nanoparticles, the functionalised group on DNA may be changed to attach to it. Numerous nanoparticle (NP)/DNA conjugates, which can be used in the sensing system of the invention, have been reported (Samanta, A.; Medintz, I. L., Nanoparticles and DNA—a powerful and growing functional combination in bionanotechnology. Nanoscale 2016, 8 (17), 9037-9095) and examples of these are given in the table below.

| Nanoparticle | Constituents | DNA |
|---|---|---|
| AuNP | Au | FAM labeled T-rich DNA |
| AuNR | Au | Leukemia T cell targeting |
| | | SH-DNA |
| Au/Ag hybrid | Au/Ag | Cytosine rich ssDNA |
| AgNP | Ag | SH-DNA |
| AgNC | Few Ag atoms | G-rich cocaine binding |
| | | aptamer |
| MNP | Fe₃O₄ | Thrombin binding |
| | | SH-DNA aptamer |
| PtNP | Pt | SH-DNA |
| PdNP | Pd | Thiol and amine |
| | | functionalized oligos |
| QD | CdSe/ZnS | Dye-labeled photonic |
| | | wire |
| SWCNT | Carbon | Ce6 conjugated |
| | | thrombin binding |
| | | aptamer |
| GO | Carbon | Short dsDNA with |
| | | random sequence |
| Micelle | DNA + PPO | ssDNA covalently |
| | | attached to PPO |
| Polyacrylamide-NP | Polyacrylamide | Dye-quencher labeled |
| | | ATP aptamer |
| Viral NP | Bacteriophage | Jurkat leukemia T cell |
| | MS2 capsid | specific DNA aptamer |
| Ferritin NPs | hFTN-H/eGFP | Amine modified PDGF |
| | or DsRed) | specific aptamer |
| UCNP | Yb³⁺ or Tm³⁺ | Amine-DNA with |
| | doped NaYF₄ | targeting sequence |
| Chalcogenide-NP | CuS | Amine modified |
| | | targeting DNA |
| Alkaline earth | Ca(H₂PO₄)₂, | eGFP encoding |
| metal NP | CaHPO₄ | plasmid DNA |
| | Ca₃(PO₄)₂ | |
| DNANP | DNA | >100 short oligomers, |
| Silane functionalised | SiO2/Silane | Amine modified DNA |
| SiO2 NP | | |

Abbreviations used in the table are as follows:

AuNP Gold nanoparticle

AuNR Gold nanorod

AgNP Silver nanoparticle

AgNC Silver nanocluster

MNP Magnetic nanoparticles

PtNP Platinum nanoparticle

PdNP Palladium nanoparticle

QD Quantum dot

SWCNT Single wall carbon nanotube

GO Graphene oxide

UCNP Upconversion nanoparticle

DNANP Deoxyribonucleic acid nanoparticle

In the present invention, the nanoparticle is typically a gold nanoparticle (AuNP). However, any of the other nanoparticles (NPs) described in the above table may alternatively be used.

There are many kinds of nanopore systems, including biological nanopore (e.g. alpha-hemolysin, MspA porin) and solid-state nanopore (e.g. Ion beam or electron beam drilled silicon nitrite membrane or graphene). In the Examples herein, nanopipettes were used among a variety of solid-state nanopore because of several advantages such as ease of fabrication, ease of set up (i.e., they can be tuned accurately) and low electrical noise. Nanopipettes may be manufactured by any suitable method available to the trained person. Quartz nanopipettes are particularly preferred as they are relatively easy to fabricate and do not introduce extra electrical noise or optical background.

Voltage-driven translocation through the nanopore may be achieved via any suitable means.

The ion currents of translocation are measured with the high-bandwidth amplifier such as a VC100 (Chimera Instruments). Typically, a grounded Faraday cage will be used to protect the nanopore system. For recording, a typical sampling rate is 1 MHz, and typically the data is filtered with 100 kHz low pass filter.

The inventors have designed a dumbbell-shaped nanoparticle dimer which is easy to fabricate and will cause specific signal shape when it passes through the nanopore. Ideally, a double peak event will be observed when a dimer molecule goes across the nanopore because the ionic signal will change while one nanoparticle is going into the pore and leaving. The dimer carrier will bind to the target protein molecule specifically by its aptamer branch. With the oligonucleotide sequences, aptamers are able to bind to their targets in a very specific way. Particularly, aptamers can be made to be applicable for almost any given target molecule, since aptamers can be collected through exponential enrichment process, in which ligands involved systematic evolution. Also, aptamers have other advantages such as low immunogenicity, small size, ease of modification and production and low toxicity. When a protein is transported by this dimer carrier, the changes of the double-peak event will give the information of this target molecule.

In one case, the nanoparticle monomer was based on AuNPs with 16-20 nm diameter with the surface functionalized by single stranded DNA (25-100 bases). However, nanoparticles of a variety of sizes may be used in the present invention. An AuNP dimer is 2 AuNP linked by a double stranded DNA. This dumbbell shape molecule can be self-assembled from two AuNP monomers with complementary ssDNA. The AuNP dimer protein carrier is a dimer with an aptamer branch in the middle. This dimer may be self-assembled from monomers with a ssDNA 1 (100 bases) and a ssDNA 2 (50 bases, DNA 2 is complementary with the end of DNA 1). The aptamer with DNA 3 (10-20 bases, DNA 3 is complementary with the unpaired part of DNA 1) then attached to the dimer. The resulting AuNPs dimer with a branch of aptamer can be the molecular carrier of a specific protein.

In an alternative embodiment, one nanoparticle (such as an AuNP) is functionalised with ssDNA with a part of aptamer sequence at the end while the other nanoparticle (such as an AuNP) is attached to ssDNA with the rest of the aptamer sequence; when the target protein is present, the two ssDNA parts of the aptamer will link together and form an aptamer which binds to the target protein, leading to the conjugation of the nanoparticle monomers. In this embodiment, the aptamer could be split in any proportion, as long as the parts link together to form a complete aptamer when the target protein is present.

It can therefore be seen that the aptamer may already be present in the DNA that is attached to one of the nanoparticles. Alternatively, the aptamer may be formed when the dimer forms, by hybridization of complementary single-stranded DNA that is bound to each of the nanoparticles.

By using the nanoparticle monomer probe, the system can be further extended to detect even smaller targets which are highly likely undetectable in single molecule level. Based on the result that the nanopore detection set up can distinguish nanoparticle monomers and dimers efficiently, the single molecule detection of small molecules can be achieved indirectly. Driven by the addition of target antigens, two nanoparticle monomers with different antibodies (both antibodies can bind to the antigen) will assemble to dimer by the strong interaction of antigen-antibody. To calculate the ratio of the double-peak signal caused by dimer molecule translocation to the single peak signal caused by monomer translocation, we can know the accurate concentration of the targeting molecule.

To detect an antigen, a pair of nanoparticle monomers (such as AuNP monomers) can be prepared in which one of the nanoparticle monomers (such as an AuNP monomer) links or is conjugated to antibody 1 while the other links or is conjugated to antibody 2 (Antibody 1 and 2 can bind to the antigen simultaneously). Once the target antigen added to the solutions containing probes with corresponding antibodies, dimer molecules will be generated by the antigen-antibody interaction. One or more of the antibodies may be modified, for example thiol functionalized.

Although antibodies are exemplified herein, it will be apparent that any antigen specific binding molecule that can be attached to a nanoparticle could be used in the present invention. In another embodiment, a pair of nanoparticle monomers (such as AuNP monomers) is prepared in which one of the nanoparticle monomers is functionalised with a ssDNA which includes a sequence which is complementary to the sequence of one end of the analyte, and the other nanoparticle monomers is functionalised with a ssDNA which includes a sequence which is complementary to the sequence of the other end of the analyte, such that in the presence of the analyte a dimer is formed. The two ssDNAs may include half of the complementary sequence each, or there may be an alternative arrangement in which, say the two ssDNAs include 40% and 60%, 30% and 70%, or any other percentage of the complementary sequence, as long as the nanoparticles dimerise on binding the target analyte. It can therefore be seen that the two nanoparticles dimerise via hybridisation of the ssDNAs attached to each of the nanoparticles to the target analyte, which is a nucleic acid. This embodiment is useful for detection of nucleic acid analytes, such as DNA or RNA, which may be single-stranded or double-stranded with a single-stranded overhang to allow for binding to the complementary sequence attached to the respective two nanoparticles. This embodiment is especially useful where the analyte is miRNA, but the analyte may be any kind of RNA such as mRNA, tRNA, siRNA, gRNA, ncRNA, exRNA, crRNA, or lncRNA.

While being the oldest type of nanopores, natural protein channels are still appealing because of several unique advantages such as high sensitivity and high resolution. However, biological nanopore biosensors are limited by their size, which is small and not tuneable. Thus, selective sensing of biomarkers in biological nanopore is still challenging.

The second aspect of the invention harnesses the potential of using nanopore based sensing techniques in biological nanopores. Inspired by the sensing of dimerization of nanoparticle probes, ssDNA and nanoparticle probes are designed. For single-molecule protein detection, a ssDNA with aptamer end and nanoparticle (such as AuNP) with antibody are used, see FIG. 13. With the presence of target protein, the ssDNA will link to the nanoparticle (such as AuNP) by an aptamer-protein-antibody bridge, FIG. 13a. In this case, the ssDNA act as a capture domain and the nanoparticle contributes to the signal amplification. The bacterial protein pore α-haemolysin (α-HL) is one of the most widely used biological channel in nanopore analytics. A ssDNA can translocate through α-HL nanopore (FIG. 13a) whereas protein cannot (FIG. 13b). However, the second level of protein blockade signal, which is highly dependent on the size and charge of the protein, may not be obvious. Herein, the nanoparticle (such as AuNP) can amplify this selective binding signal, see FIG. 13.

Based on the same principle, miRNA also can be detected by ssDNA and nanoparticle (such as AuNP) with specific sequence (FIG. 14). With the presence of the target miRNA, the ssDNA carrier with a complementary DNA (cDNA1) end to miRNA and the nanoparticle probe modified with the remainder of the complementary DNA (cDNA2) will self-assemble to a ssDNA-miRNA-nanoparticle complex due to DNA-RNA hybridisation. When free ssDNA translocates through the biological nanopore, the ionic current will be blocked by the DNA molecule and show a drop-down. Since the hybridised dsDNA cannot enter the nanopore, for ssDNA bound with target miRNA, the signal will show a subpeak due to the stuck or release of target miRNA. With the form of ssDNA-miRNA-nanoparticle complex, the subpeak will be amplified due to the existence of the nanoparticle (such as AuNP). The nanoparticle serves as a signal amplifier and effectively increases signal-to-noise ratio.

Embodiments described herein in relation to the first aspect of the invention are applicable to the second aspect of the invention mutatis mutandis.

The present invention will be further understood by reference to the following examples.

### Examples

### Materials & Methods

### The Fabrication of Nanopipettes

The single barrel quartz capillaries (o.d., 1.0 mm, i.d., 0.7 mm, Intracell) were plasma cleaned (Harrick Plasma), and pulled with a laser-based P-2000 pipette puller (Sutter Instruments) using a two-line program (heat 800, filament 4, velocity 30, delay 170, and pull 80; heat 825, filament 3, velocity 20, delay 145, and pull 130) to produce nanopipettes with the nanopore diameters of approximately 34 nm at the tip as characterised by SEM imaging. It should be noted that the above pulling parameters are instrument specific and variations will exist from puller to puller.

### The Fabrication of AuNP Based Nanostructures

The effective length of a nanopipette is the portion of the electrolyte-filled pore over which the majority (for our calculations 75-80%) of its ionic resistance is focused. In addition, the voltage drop is greatest in this area, resulting a strongest electric field. For a cylindrical nanopore, the pore length (On the solid state nanopore always been considered as the thick of the membrane) equals to the effective length of the nanopore. Theoretically, the voltage drops linearly along the pore length in these cylindrical pores. However, the conical nanopore, which located on the tip of the nanopipette, results a nonlinear drop of the electric field and resistance along with the pore length because the pore radius changes along the distance to the end.

To detect the nanopipette effective length, the resistance distribution along the pore axis should be analysed (Figure. 5). The resistance with different position (Rx) of the nanopore can be estimated. The distance from the end, x, is ranging from 0 to L, which is a length that long enough and can be studied from the SEM images. D0 represents the diameter of the end of the nanopipette part and the DL represents the diameter of the area which L from the end. The diameter along with the distance, Dx, can be calculated. Then the Rx can be estimated.

Dx can be expressed as the following function of x:

$${D}_{x} = \frac{x ( {D}_{L} - {D}_{0} ) + L {D}_{0}}{L} \qquad ( 1 )$$

In our calculation, the step of x is 1 nm.
The resistance Rx can be calculated as following function:

$${R}_{x} = \frac{4 L ρ}{π {D}_{0} {D}_{x}} \qquad ( 2 )$$

where p is the specific resistance of electrolyte which fills the pore. The Rₜₒₜ represents the total resistance.

### The Fabrication of Thrombin-Binding Aptamer (TBA) Modified AuNP Dimers

The 17 nm AuNPs were concentrated 10-fold and resuspended in 0.5×TBE buffer to a final concentration of 10 nM. The prepared AuNPs were then mixed with the ssDNA5 (10 μM, dissolved in TE buffer), in a DNA:AuNP molar ratio of 5:1. NaCl solution (5 M) was added to the mixture to a final NaCl concentration of 50 mM, and left at 25° C. for 12 h. Finally, the mixture was centrifuged (three times) at 7000 rpm for 10 min to remove the excess DNA, and resuspended in TBE buffer. The process of AuNPs (17 nm) modified with the ssDNA6 were same as DNAS. 100 μL of AuNP-DNAS and 100 μL of AuNP-DNA6 were mixed in TBE buffer containing 50 mM NaCl. The mixture was hybridised for 12 h at room temperature with gentle stirring. The mixture was centrifuged at 5000 rpm for 10 min to remove the single nanoparticles and collect the AuNPs dimer thrombin carrier. The sequence of DNA is shown in FIG. 6.

### The Fabrication of the Monomer PCT Probes

2 mL AuNPs (20±3 nm) were centrifuged for 10 min at 8000 rpm and then resuspended in 200 μL of 10 mM phosphate buffer (PB) solutions, which was adjusted to pH 9 with 0.1 M K2CO3. Next, 100 μL of the AuNPs were conjugated with anti-PCT mAb (10 μL, 100 μg/m L) and the other 100 μL AuNPs were modified with anti-PCT smAb (10 μL, 100 μg/mL), respectively. Then, the solutions were blocked by BSA (10 μL, 500 μg/mL) for 30 min after the incubation of 1 h. Next, the functionalized AuNPs solutions were centrifuged for 10 min at 7500 rpm so that the final product can be collected as the monomer PCT probes.

### Nanostructures Translocation Experiment

The translocation experiments were performed from inside nanopipette to outside, where the AuNPs based nanostructures are loaded in the nanopipette which is the cis chamber as well as an Ag/AgCl working/patch electrode while an Ag/AgCl counter/reference electrode was placed in the blank buffer located in the bath as the trans chamber. The buffer used in the translocation experiments consisted of 50 mM KCl. 10 mM Tris-EDTA (pH=8) unless reported otherwise. For the binding assay of thrombin or PCT by using corresponding strategies, 1 nM thrombin carriers or PCT probes are incubated with target analytes at a different concentration at least 2 hrs. The buffer containing target analytes were filled inside the nanopipette along with the electrode. Then a voltage was applied by a high bandwidth amplifier VC100 (Chimera Instruments) between the electrodes on both sides of the nanopore, and the current-time trace can be recorded. The data was then resampled to 1 μs and refiltered to 100 kHz and was analysed by a customised Matlab App.

### Results

### Quantification of AuNP Monomers, Dimers, and Trimers

To demonstrate the screening ability of the AuNP conjugates, we quantified the resolution of our platform. First, we confirmed the ability to differentiate between monomers and dimers linked with differing DNA lengths. AuNP monomers were fabricated by attaching a thiol-modified 25-mer ssDNA to the surface of 17 nm in diameter AuNPs (FIG. 2a). AuNP symmetrical dimers were fabricated by self-assembly of two AuNP monomers with one consisting of a 15 bases complementary sequence. To challenge the spatial resolution, we fabricated two kinds of AuNP symmetrical dimers: one with 35 bases linkers and the other with 115 bases linkers. Asymmetrical dimers we also designed consisting of 10 nm AuNP monomers and 20 nm AuNPs. Finally, trimers were also quantified and could be achieved by controlling the NP to DNA ratio.

### The Fabrication of AuNP Based Nanostructures

### AuNP Monomer

The synthesised AuNPs (17 nm) were concentrated 10-fold and resuspended in 0.5×TBE buffer to a final concentration of 10 nM. The prepared AuNPs were then modified with the ssDNA1 (10 μM, dissolved in TE buffer), in a DNA:AuNP molar ratio of 5:1. NaCl solution (5 M) was added to the mixture to a final NaCl concentration of 50 mM, and left at 25° C. for 12 h. Finally, the mixture was centrifuged (three times) at 7000 rpm for 10 min to remove the excess DNA and resuspended in TBE buffer. The schematic of AuNP monomer is shown in FIG. 7a.

### AuNP Symmetrical Dimer (35 Bases Linker)

The process of AuNPs (17 nm) modified with the ssDNA2 were same as DNA1. 100 μL of AuNP-DNA1 and 100 μL of AuNP-DNA2 were mixed together in TBE buffer containing 50 mM NaCl. The mixture was hybridized for 12 h at room temperature with gentle stirring. The mixture was centrifuged at 5000 rpm for 10 min to remove the single nanoparticles and collect the AuNP symmetrical dimers with short linker. The schematic of AuNP symmetrical dimer (35 bases linker) is shown in FIG. 7b.

### AuNP Asymmetrical Dimer

AuNPs were concentrated 10-fold and resuspended in 0.5×TBE buffer to a final concentration of 10 nM. The prepared 20 nm AuNPs were then modified with the ssDNA1 (10 μM, dissolved in TE buffer, mixture1) and 10 nm AuNPs were then modified with the ssDNA2 (10 μM, dissolved in TE buffer, mixture2), in a DNA:AuNP molar ratio of 5:1. NaCl solution (5 M) was added to the mixture to a final NaCl concentration of 50 mM, and left at 25° C. for 12 h. Finally, the mixture1 was centrifuged (three times) at 7000 rpm for 10 min, and the mixture2 was centrifuged (three times) at 8500 rpm for 10 min to remove the excess DNA and resuspended in TBE buffer. 100 μl of 20 nm AuNP-DNA1 and 100 μl of 10 nm AuNP-DNA2 were mixed together in TBE buffer containing 50 mM NaCl. The mixture was hybridized for 12 h at room temperature with gentle stirring. The mixture was centrifuged at 5000 rpm for 10 min to remove the single nanoparticles and collect the AuNP asymmetrical dimers. The schematic of AuNP asymmetrical dimer is shown in FIG. 7c.

### AuNP Trimer

100 μl of 17 nm AuNP-DNA1 and 200 μL of 17 nm AuNP-DNA2 were mixed together in TBE buffer containing 50 mM NaCl. The mixture was hybridized for 12 h at room temperature with gentle stirring. The mixture was centrifuged at 4000 rpm for 10 min to remove the single nanoparticles and collect the AuNPs trimers. The schematic of AuNP trimer is shown in FIG. 7d.

### AuNP Symmetrical Dimer (115 Bases Linker)

The 17 nm AuNPs were concentrated 10-fold and resuspended in 0.5×TBE buffer to a final concentration of 10 nM. The prepared AuNPs were then modified with the ssDNA3 (10 μM, dissolved in TE buffer), in a DNA:AuNP molar ratio of 5:1. NaCl solution (5 M) was added to the mixture to a final NaCl concentration of 50 mM, and left at 25° C. for 12 h. Finally, the mixture was centrifuged (three times) at 7000 rpm for 10 min to remove the excess DNA, and resuspended in TBE buffer. The process of AuNPs (17 nm) modified with the ssDNA4 were same as DNA3. 100 μL of AuNP-DNA3 and 100 μL of AuNP-DNA4 were mixed together in TBE buffer containing 50 mM NaCl. The mixture was hybridized for 12 h at room temperature with gentle stirring. The mixture was centrifuged at 5000 rpm for 10 min to remove the single nanoparticles and collect the AuNP symmetrical dimers with long linker. The schematic of AuNP symmetrical dimer (115 bases linker) is shown in FIG. 7e.

### 4-ATP Labelled AuNP Symmetrical Dimers (SERS Sample)

The 35 nm AuNPs were concentrated 10-fold and resuspended in 0.5×TBE buffer to a final concentration of 10 nM. The prepared AuNPs were then modified with the ssDNA1 (10 μM, dissolved in TE buffer), in a DNA:AuNP molar ratio of 5:1. NaCl solution (5 M) was added to the mixture to a final NaCl concentration of 50 mM, and left at 25° C. for 12 h. Finally, the mixture was centrifuged (three times) at 5000 rpm for 10 min to remove the excess DNA and resuspended in TBE buffer.

Before modified with the ssDNA2, 4-Aminothiophenol was added to the 35 nm AuNPs, with the final concentration of 10 μM. After 6 h, the AuNPs were centrifuged at 5000 rpm for 10 min to remove the unconnected 4-Aminothiophenol. Then, the above AuNPs were modified with ssDNA2. Finally, 100 μl of 35 nm AuNP-DNA1 and 100 μL of 35 nm AuNP-DNA2 were mixed together in TBE buffer containing 50 mM NaCl. The mixture was hybridized for 12 h at room temperature with gentle stirring. The mixture was centrifuged at 3000 rpm for 10 min to remove the single nanoparticles and collect the 4-ATP labelled AuNP symmetrical dimers. In order to increase the stability of the large AuNPs in high salt concentration, the SERS samples are further modified by PEG-SH. The schematic of 4-ATP labelled AuNP symmetrical dimers is shown in FIG. 7f.

The geometry of the nanostructures was confirmed by transmission electron microscopy (TEM), FIG. 2. To avoid the unpredictable orientation of the conjugated molecules with high viscosity during translocation, all AuNPs based samples were not protected by polyethylene glycol (PEG) unless otherwise stated. All NP conjugates were dispersed after fabrication and measured by UV-Vis after 24 hours stabilisation in the buffer 50 mM KCl, 10 mM Tris-EDTA and used in nanopore experiments. This ionic strength was chosen to minimise NP aggregation and at the same time maximise the signal to noise of the measurements.

Nanopore experiments were done using single-barrelled nanopipettes^{39, 40}, which can be fabricated by laser-assisted pulling of quartz capillaries. The nanopores were pulled with an average diameter slightly larger than the dimensions of the nanoparticles, with and average diameter of 34±5 nm, as measured by scanning electron microscope (SEM). These dimensions closely matched the diameters estimated from nanopore conductance measurements ( ) of 15.3±2.4 nS in 100 mM KCl (n=18). Using SEM imaging, the taper angle of the nanopipette tip was measured to be 16.9±1.1° over the first 100 nm (n=18), which allowed us to estimate an effective sensing length between 25 to 50 nm, based on a 75-80% resistance drop at the nanopore. The analyte was filled inside the nanopipette where a patch electrode (AgCl) was placed, and a ground/reference electrode (AgCl) was placed in a bath, outside the pipette. By applying a negative potential, it was possible to transport the AuNPs from inside (cis) to outside (trans) of the nanopipette. A high bandwidth amplifier (Chimera VC100) was used with a 1 μs sampling rate and 100 kHz low-pass digital filter.

FIG. 2 shows a comparison of translocation characteristics between the different conjugates. The simplest constructs, AuNP monomers, translocated relatively quickly in a single file translocation with a with a single peak dwell time distribution (mean dwell time of 8.3±1.4 μs at an applied potential of −600 mV), FIG. 2a. In comparison symmetric and asymmetric dimers with a 35 base DNA spacer, FIG. 2b-2c exhibited clear double file events. The current amplitudes of each subpeak are consistent with the size of the individual AuNPs within the conjugate. The observations are consistent with the model that the first peak appears due to the negatively charged AuNPs passing though the nanopore sensing area. This is followed by a decrease of the current due to the reduced charge on the linker. Finally, the 2^{nd} AuNP results in a 2^{nd} peak. The mean dwell time of the dimers in FIG. 2b is 20.8±2.7 μs which is just over twice the monomer translocation time and is consistent with linear transport through the nanopore. Importantly, one could distinguish between the peaks with high spatial resolution, as the distance between the peaks was a mere 28 nm. Furthermore, it was possible to differentiate between spacers of different lengths, which correspond to distance of 28 and 51 nm, respectively.

When asymmetric AuNPs dimers were translocated out of the nanopipette (FIG. 2c), their asymmetric size was reflected in the shape of individual event in current time traces as well as the corresponding current peak distributions. The double peak current distributions were recorded with high peak currents of 155.51±21.55 pA and 81.83±13.47 pA, corresponding to translocations of 20 nm and 10 nm AuNP, respectively. Interestingly, nearly 95% of all translocation events of asymmetric dimers showed a preferential orientation with the larger NP being transported first, which was attributed to the larger NPs carrying higher surface charge. Although this was not the focus of the present manuscript, we also investigated the possibility of translocating and detecting NP trimers, FIG. 2d. Trimers could be detected with well-defined individual monomer signatures and it took 32.7±5.0 μs for individual trimers to translocate through the nanopore, which is 3.94-folds (4.23-folds for spatial length) longer than the monomer dwell time and 1.57-folds (1.61-folds for spatial length) longer than the dimer dwell time, respectively.

To further characterise the translocations of these metallic nanoparticles, single particle surface-enhanced Raman scattering (SERS) was also performed on a modified gold coated (10 nm thickness) nanopipette. Due to the coupling and proximity between dimer and metal pipette surface, this results in a significant increase in Raman signal.^{41, 42} To achieve single-particle SERS somewhat larger 35 nm AuNP symmetrical dimers we used due to the higher scattering cross-section. The AuNPs were functionalized with 4-Aminothiophenol (ATP) dye (FIG. 2e, FIG. 7). The dimer is further stabilized with PEG to ensure the particles do not aggregate at the 100 mM salt concentrations required to perform the translocations. A typical SERS spectrum of the NPs in bulk solution is shown and consists of expected peaks at 1138, 1387 and 1571 cm⁻¹ (FIG. 8). This is comparable to the data obtained for single particle SERS as can be seen from the transients in FIG. 2e. The integration time for the transients was 810 μs, and translocations were obtained at −800 mV. In this example, the optical translocation times were 3.24 ms which is longer than the corresponding electrical events (0.79±0.29 ms). This is due to the nanopore sensing region being much smaller than the diffraction limited laser spot size (ca. 1 μm). As a negative control, Raman spectra, which shows no Raman signal, were also acquired for the event when the reverse potential is applied to diffuse the dimer away from the nanopore. We envisage that this method can also be used to perform molecular assays and complements the electrical work shown in this manuscript.

The Design of AuNPs Dimer Thrombin Carrier and the Sensing of it with Thrombin

The first strategy for the biosensing is to utilise the dimer molecule as a carrier to drive and detect specific protein molecules. The AuNP dimer protein carrier, which is based on AuNP symmetrical dumbbell system with a DNA bridge, was engineered to contain a part of ssDNA overhang with the specific aptamer sequence. Due to the high affinity and selectivity of the aptamer and protein interaction, the dimer protein carriers with specific aptamer will only bind corresponding proteins in trace level, FIG. 3a.

In this case, human alpha-thrombin (α-thrombin; M.W. 37.5 kDa; pI of 7.0-7.4), a multifunctional protease in the bloodstream, became our target due to its significant roles in various crucial physiological and pathological processes, such as blood coagulation, thrombosis and angiogenesis. It is essential to detect thrombin at a trace amount with high sensitivity. However, using plain solid-state nanopores to sense this biomarker with such small size and heterogeneous charge, is difficult to achieve. Therefore, AuNPs dimer thrombin carrier was designed to pave the way of thrombin detection at the single-molecule level.

The thrombin-binding aptamer (TBA), which binds to thrombin selectively and compactly (K_{d}^{˜}35-100 nM in solid phase assays³⁶), is a 15-mer (5′-GGTTGGTGTGGTTGG-3′—SEQ ID NO: 10) ssDNA folding into stable intramolecular G-quadruplex in the presence of K⁺.^{43, 44} The TBA sequence was anchored on a protuberance of the dimer 115-mer DNA bridges as a binding site of thrombin located in the middle of the dumbbell molecules, FIG. 6. Prior to nanopore measurements, the efficiency of the binding between thrombin and the corresponding carriers was confirmed by UV-vis. By adding 4 nM thrombin to 1 nM, AuNP dimer thrombin carrier dispersions with 50 mM KCl and 10 mM Tris-EDTA at pH 8, the AuNPs based molecules started oligomerisation due to the selective binding weakening the holistic charge of the molecule. As a control, the pristine AuNPs dimer molecules in the same ionic strength would not aggregate until the concentration of thrombin reached 100 nM, which masked the surface of the dimer molecules.

As a control, we first examine the translocation of TBA modified AuNP dimer, FIG. 3b. Comparing the translocation events of unmodified AuNPs dimer (FIG. 10) and TBA modified AuNPs dimer (FIG. 3b) with same particle size and same linker length, the difference in dwell time, peak current, and fraction position are negligible. With the addition of 1 nM thrombin into 1 nM TBA modified AuNPs dimer, some triple peak events were observed when these complexes were driven by −600 mV potential, FIG. 3c. Unlike the triple peaks of the AuNPs trimers, the triple peaks events of the protein bound dimer revealed a distinct signature that the second peak (located on 0.51 of the normalised peak position) always smaller than the first and third peak (located on 0.22 and 0.86 of the normalised peak position, respectively). As discussed before, the peaks are generated when the nanopore conductance changed by the different part of the nanostructures. In the pH 8 environment, the thrombin and TBA part are negatively charged which is ascribable to the deprotonation of the amino acid, although it is not comparable with the charge of the AuNPs. Therefore, the small enhancement peak, with an 80% magnitude of the AuNPs peaks, occurred at the middle of the events, corresponding to the thrombin anchored DNA bridges. However, proved by the UV-Vis, the holistic charge of the molecular carrier is weakened after the addition of thrombin, which leading a 1.1-fold increase of the translocation dwell time, FIG. 10.

By counting the percentage of the triple peak events of all triple peak and double peak events, the binding ratio can be calculated, FIG. 3d-e. As expected, the proportion of triple peak signature raised with the addition of thrombin. In this experiment, we varied the concentration of thrombin from 0 to 2 nM while the concentration of the dimer remained 1 nM. From the statistics of normalised peak position (FIG. 3d), it is evident that there are no triple peak events without the addition of thrombin and then the middle peak showed stepwise with the increasing of the thrombin concentration. The subtle change of the concentration of thrombin can be monitored as approximate 50 pM change of the thrombin; the triple/double peak ratio will change 1%. Further, to validate the selectivity of the AuNP dimer thrombin carrier, 1 nM lysozyme was incubated with 1 nM thrombin carrier and subsequent nanopore detection of the mixture was performed, FIG. 11. There is no sign of triple peak signature but the double peak events, which are corresponding to the unbounded dimer carrier.

### The Design of AuNP Monomer PCT Probes and the Detection of PCT in Single Molecule Level

The AuNP dimer protein carriers showed the ability to sense corresponding protein with high sensitivity and selectivity. However, the aptamers modified carriers, including most carriers reported previously, cannot sense smaller targets (<15 kDa). What is worse, with the decreasing of the biomolecule size, the signatures become progressively hard to detect due to the lowering of the signal-to-noise ratio.

Herein, based on our previous work^{45,46}, a universal strategy of sensing small antigen molecules has been performed. In detail, half of AuNPs are modified by the corresponding antibody (mAb) of the target antigen while the rest are modified with complementary secondary antibody (smAb). With the presence of the antigen, the monomers will self-assemble to dimer with a ‘sandwich’ formation (FIG. 4a) and this changing can be recorded when the molecules pass through the nanopore.

In this case, we use the AuNP monomer antigen probes to detect procalcitonin (PCT; M.W. 12.8 kDa; pI of 6.5) which is a peptide precursor of the hormone calcitonin. Due to the PCT level variance between healthy and microbial infected individuals, it has become an important biomarker to improve bacterial infections identification and guide antibiotic therapy. A pair of AuNP monomer PCT probes were fabricated to sensing PCT in single-molecule level, which cannot be studied by the conventional translocation due to the extremely small size. To ensure the intramolecular nanostructure can be distinguished by the nanopore in this case, we increased the AuNP size to 20 nm. Therefore, with an antibody-antigen-antibody sandwich linker which is approximately 10 nm, the substructure of the dumbbell molecule (the centre of one AuNP to the other is around 30 nm) can be distinguished by nanopores with sub-30 nm effective length.

A comparison between the translocation of 2 nM AuNP monomers (50% are functionalized by PCT mAb, and 50% are functionalized by smAb) and the assembled dumbbell complex after adding 20 nM PCT, was shown in FIG. 4b-c. Without the presence of PCT, no double peak signal but a single peak signal was observed during the translocation of 2 nM AuNP monomers with 50 mM KCl, pH 8. With the addition of 20 nM PCT, about 15% of the single peaks transformed to clear double peak events. Each peak is the result of the translocating of AuNP whereas the trough is due to the translocation of the weak and uniform charged sandwich linker (PI of mAb, smAb is 6.6-7.2). As a negative control, no double peak signatures observed when PCT was replaced by other antigens, for example, insulin, FIG. 12.

To validate the sandwich immunoassay mode can be used in clinical diagnosis level, the nanopore sensing studies of AuNP monomer probes with different concentration of PCT was performed. In this case, the concentration of the probes was kept as 2 nM while the concentration of PCT was ranging from 0 to 200 nM. As expected, the percentage of the single peak (located on 0.49 of the normalised peak position) decreased whereas the proportion of double peak signatures (located on 0.30 and 0.78 of the normalised peak position) raised with the increasing of PCT, FIG. 4d(i). The results were further confirmed by TEM (FIG. 4d(ii)), which provided visualised evidence that the proportion of dimer increased with more addition of PCT.

In bacterial infections, sepsis, severe sepsis and septic shock, PCT in plasma concentrations increases from 0.15 to more than 10 ng/ml. This increase often correlates with the severity of the disease and with mortality. At the same time, PCT has also been used to guide antibiotic therapy, for example, if PCT level <0.1 ng/ml, antibiotic therapy is strongly discouraged; if PCT level >1 ng/ml, antibiotic therapy is strongly encouraged. With high sensitivity, the PCT monomer probe is capable of sensing this important biomarker in this range (FIG. 4e inset).

Molecular Probes for Single-Molecule Detection of miRNA

miRNA are a class of short non-coding RNAs that function in RNA silencing and post-transcriptional gene regulation. Besides their participation in regulating normal physiological activities, specific miRNA types could act as oncogenes, tumor suppressors, or metastasis regulators, which are critical biomarkers for cancer. Conventional methods include Northern blotting, in situ hybridization, RT-qRCR, or microarrays. However, these methods require sample preparation or processing. In addition each technique has specific limitations such as low throughput and low sensitivity (for northern blotting), semi-quantitative (for in situ hybridization), time consuming, critical reaction condition (for RT-qPCR). Recent advances in nanopore technology offer the promise of addressing some of these drawbacks for detection of miRNA with high sensitivity and selectivity.[47] However, the signal of these short fragments (typically 18-23 bases) is hard to detect directly with solid-state nanopores due to the high-speed translocation and low signal-to-noise ratio, FIG. 16. Here, we use AuNP dimer self-assembly to amplify this translocation signal, leading to very efficient miRNA detection at the single-molecule level.

In this study, we use AuNP molecular probes for the detection of miRNA-141. miRNA-141 is commonly dysregulated in malignant tumors such as those associated with prostate cancer and plays essential roles in tumor development and progression, becoming a powerful potential biomarker of prostate cancer.[48] Prostate cancer is the second most common cancer in men worldwide; however, disease outcome is difficult to predict in large part due to the lack of efficient diagnostic strategies. As such, miRNA-141 has the potential to become a useful biomarker.

The molecular probes consisted of two populations of ssDNA functionalized to AuNP monomers. Each of them was modified by an 11 base recognition chain, which can hybridize with half of the 22-base-long miRNA-141, FIG. 17. With the addition of the target, the monomer probes self-assemble to form dimers and produce doublet signatures, FIG. 15a-c. A binding assay was performed within the miRNA concentration range of 1 pM to 100 nM. As previously shown for PCT, the number of dimers, and hence doublets, increases with concentration, FIG. 15d-e. Dimer formation is validated and compared with TEM, FIG. 18, providing visual evidence of dimer formation due to the presence of miRNA-141. Typically, the concentration of miRNA-141 between fM and pM in unprocessed prostate cancer patient samples, and between pM to nM in extracted miRNA samples.

The specificity of the molecular probes was verified by detecting miRNA-200a, which is also in the miR-200 family and share seed sequences differing in only two nucleotides when compared with miRNA-141, FIG. 19. The full recognition of miRNA-141 gives a significant binding result, whereas the control experiment, which is detecting the miRNA-200a, leads to a low value of the binding ratio, FIG. 15e. The result is further confirmed by TEM, FIG. 15e, FIG. 20. Such high selective capability probably benefits from the dimerization mechanism. For example, for miRNA-141, the monomer probes can be linked to the dimer because the ssDNA is fully matching the target. In contrast, for the miRNA-200a, the two mismatch points happened on the same ssDNA of one monomer probe, leading to a very low binding affinity, which causes unsuccessful dimerization. This result shows that the AuNP monomer probe can detect the target with high specificity.

### CONCLUSION

Although nanoparticle-based superstructures have already been reported and some of them are detected by nanopore sensing49, many of these tests are based on sensing the exclude volume rather than the substructures of these nanoparticle conjugates, leading to false positive in the nanoparticle based sensing applications. We demonstrate that it is possible to use a finely tuned nanopore testing platform incorporating with high-bandwidth instruments to depict the sub-structure of these molecules. We have shown this set-up can differentiate AuNP monomer, AuNPs symmetrical or asymmetrical dimer, and AuNPs trimer. By utilising the plasmonic effect of the dimer system, the single molecule SERS detection was also applied. Based on these validations of the detection resolution, two strategies of sensing biomolecules in a single molecule level are performed.

In summary, the first strategy is to use an AuNPs dimer protein carrier, which is an AuNPs dumbbell system incorporating with an aptamer prominence on the middle of the DNA linker, to detect proteins. The second strategy is to utilise the feature of the self-assembling of AuNP monomer antigen probes to dimers with the addition of the specific antigens. Both strategies are fully flexible to detect a number of biomolecules with just changing the aptamer sequence or antibodies. The excellent selectivity and affinity of aptamer-protein or antibody-antigen provide the possibility to apply these strategies to diagnostics for detecting biomarkers in trace amount. Importantly, to different biomolecules, different strategy can be chosen. For example, to sense some large proteins, the aptamer modified carrier can be used because an evident signature in the middle can provide information such as the size and charge of the target. Otherwise, if the biomolecule is too small to generate the ripples on the electric signal, the other strategy, an indirect detection in single molecule level, can be applied. Both strategies are not only capable of validating the presence of the specific targets, but also can quantify the concentration of them in clinical diagnosis level.

### REFERENCES

1. Miles B N, Ivanov A P, Wilson K A, Dogan F, Japrung D, Edel J B. Single molecule sensing with solid-state nanopores: novel materials, methods, and applications. Chemical Society reviews 42, 15-28 (2013).
2. Meller A, Nivon L, Branton D. Voltage-driven DNA translocations through a nanopore. Physical review letters 86, 3435-3438 (2001).
3. Wanunu M. Nanopores: A journey towards DNA sequencing. Physics of life reviews 9, 125-158 (2012).
4. Ying Y L, Zhang J J, Gao R, Long Y T. Nanopore-Based Sequencing and Detection of Nucleic Acids. Angewandte Chemie-International Edition 52, 13154-13161 (2013).
5. Wanunu M. Nanopores: A journey towards DNA sequencing. Physics of life reviews 9, 125-158 (2012).
6. Meller A, Branton D. Single molecule measurements of DNA transport through a nanopore. Electrophoresis 23, 2583-2591 (2002).
7. Li J L, Gershow M, Stein D, Brandin E, Golovchenko J A. DNA molecules and configurations in a solid-state nanopore microscope. Nature Materials 2, 611-615 (2003).
8. Fologea D, Gershow M, Ledden B, McNabb D S, Golovchenko J A, Li J L. Detecting single stranded DNA with a solid state nanopore. Nano letters 5, 1905-1909 (2005).
9. Wanunu M, Morrison W, Rabin Y, Grosberg A Y, Meller A. Electrostatic focusing of unlabelled DNA into nanoscale pores using a salt gradient. NatNanotechnol 5, 160-165 (2010).
10. Plesa C, Kowalczyk S W, Zinsmeester R, Grosberg A Y, Rabin Y, Dekker C. Fast Translocation of Proteins through Solid State Nanopores. Nano letters 13, 658-663 (2013).
11. Li W, Bell N A W, Hernandez-Ainsa S, Thacker V V, Thackray A M, Bujdoso R, Keyser U F. Single Protein Molecule Detection by Glass Nanopores. ACS nano 7, 4129-4134 (2013).
12. Steinbock U, Krishnan S, Bulushev R D, Borgeaud S, Blokesch M, Feletti L, Radenovic A. Probing the size of proteins with glass nanopores. Nanoscale 6, 14380-14387 (2014).
13. Lan W J, Holden D A, Zhang B, White H S. Nanoparticle Transport in Conical-Shaped Nanopores. Analytical Chemistry 83, 3840-3847 (2011).
14. Tsutsui M, Hongo S, He Y, Taniguchi M, Gemma N, Kawai T. Single-Nanoparticle Detection Using a Low-Aspect-Ratio Pore. ACS nano 6, 3499-3505 (2012).
15. Lan W-J, Holden D A, Liu J, White H S. Pressure-Driven Nanoparticle Transport across Glass Membranes Containing a Conical-Shaped Nanopore. Journal of Physical Chemistry C 115, 18445-18452 (2011).
16. Davenport M, Healy K, Pevarnik M, Teslich N, Cabrini S, Morrison A P, Siwy Z S, Letant S E. The Role of Pore Geometry in Single Nanoparticle Detection. ACS nano 6, 8366-8380 (2012).
17. Wang Y X, Kececi K, Mirkin M V, Mani V, Sardesai N, Rusling J F. Resistive-pulse measurements with nanopipettes: detection of Au nanoparticles and nanoparticle-bound anti-peanut IgY. Chemical Science 4, 655-663 (2013).
18. Zahid O K, Wang F, Ruzicka J A, Taylor E W, Hall A R. Sequence-Specific Recognition of MiRNAs and Other Short Nucleic Acids with Solid-State Nanopores. Nano letters 16, 2033-2039 (2016).
19. Cai H, et al. Resistive-Pulse Measurements with Nanopipettes: Detection of Vascular Endothelial Growth Factor C (VEGF-C) Using Antibody-Decorated Nanoparticles. Analytical Chemistry 87, 6403-6410 (2015).
20. Heins E A, Siwy Z S, Baker L A, Martin C R. Detecting single porphyrin molecules in a conically shaped synthetic nanopore. Nano letters 5, 1824-1829 (2005).
21. Luan B, Stolovitzky G, Martyna G. Slowing and controlling the translocation of DNA in a solid-state nanopore. Nanoscale 4, 1068-1077 (2012).
22. Keyser U F. Controlling molecular transport through nanopores. Journal of the Royal Society Interface 8, 1369-1378 (2011).
23. Wei R, Gatterdam V, Wieneke R, Tampe R, Rant U. Stochastic sensing of proteins with receptor-modified solid-state nanopores. Nature nanotechnology 7, 257-263 (2012).
24. Ding S, Gao C L, Gu L Q. Capturing Single Molecules of Immunoglobulin and Ricin with an Aptamer-Encoded Glass Nanopore. Analytical Chemistry 81, 6649-6655 (2009).
25. Actis P, Rogers A, Nivala J, Vilozny B, Seger R A, Jejelowo O, Pourmand N. Reversible thrombin detection by aptamer functionalized STING sensors. Biosensors & Bioelectronics 26, 4503-4507 (2011).
26. Yusko E C, Johnson J M, Majd S, Prangkio P, Rollings R C, Li J L, Yang J, Mayer M. Controlling protein translocation through nanopores with bio-inspired fluid walls. Nature nanotechnology 6, 253-260 (2011).
27. Al Sulaiman D, Cadinu P, Ivanov A P, Edel J B, Ladame S. Chemically Modified Hydrogel-Filled Nanopores: A Tunable Platform for Single-Molecule Sensing. Nano letters 18, 6084-6093
28. Xue L, Cadinu P, Nadappuram B P, Kang M, Ma Y, Korchev Y, Ivanov A P, Edel J B. Gated Single-Molecule Transport in Double-Barreled Nanopores. ACS applied materials & interfaces 10, 38621-38629 (2018).
29. Ren R, Zhang Y J, Nadappuram B P, Akpinar B, Klenerman D, Ivanov A P, Edel J B, Korchev Y. Nanopore extended field-effect transistor for selective single-molecule biosensing. Nature Communications 8, (2017).
30. Ai Y, Liu J, Zhang B K, Qian S. Field Effect Regulation of DNA Trans location through a Nanopore. Analytical Chemistry 82, 8217-8225 (2010).
31. Larkin J, Henley R Y, Muthukumar M, Rosenstein J K, Wanunu M. High-Bandwidth Protein Analysis Using Solid-State Nanopores. Biophysical journal 106, 696-704 (2014).
32. Rosenstein J K, Wanunu M, Merchant C A, Drndic M, Shepard K L. Integrated nanopore sensing platform with sub-microsecond temporal resolution. Nature Methods 9, 487-U112 (2012).
33. Singer A, Wanunu M, Morrison W, Kuhn H, Frank-Kamenetskii M, Meller A. Nanopore Based Sequence Specific Detection of Duplex DNA for Genomic Profiling. Nano letters 10, 738-742 (2010).
34. Bell N A W, Keyser U F. Digitally encoded DNA nanostructures for multiplexed, single-molecule protein sensing with nanopores. Nature nanotechnology 11, 645-+(2016).
35. Sze J Y Y, Ivanov A P, Cass A E G, Edel J B. Single molecule multiplexed nanopore protein screening in human serum using aptamer modified DNA carriers. Nature Communications 8, (2017).
35a. WO 2018/189530
36. Beamish E, Tabard-Cossa V, Godin M. Identifying Structure in Short DNA Scaffolds Using Solid-State Nanopores. Acs Sensors 2, 1814-1820 (2017).
37. Loh, A Y Y, Burgess C H, Tanase D A, Ferrar G, McLachlan M A, Cass, A E G, Albrecht, T. Electric Single-Molecule Hybridization Detector for Short DNA Fragments. Analytical chemistry 23, 14063-14071 (2018).
38. Steinbock U, Otto O, Chimerel C, Gornall J, Keyser U F. Detecting DNA Folding with Nanocapillaries. Nano letters 10, 2493-2497 (2010).
39. Ying L M, White S S, Bruckbauer A, Meadows L, Korchev Y E, Klenerman D. Frequency and voltage dependence of the dielectrophoretic trapping of short lengths of DNA and dCTP in a nanopipette. Biophysical journal 86, 1018-1027 (2004).
40. Karhanek M, Kemp J T, Pourmand N, Davis R W, Webb C D. Single DNA molecule detection using nanopipettes and nanoparticles. Nano letters 5, 403-407 (2005).
41. Nam J M, Oh J W, Lee H, Suh Y D. Plasmonic Nanogap-Enhanced Raman Scattering with Nanoparticles. Accounts of Chemical Research 49, 2746-2755 (2016).
42. Cecchini M P, Wiener A, Turek V A, Chon H, Lee S, Ivanov A P, McComb D W, Choo J, Albrecht T, Maier S A, Edel J B. Rapid Ultrasensitive Single Particle Surface-Enhanced Raman Spectroscopy Using Metallic Nanopores. Nano letters 13, 4602-4609 (2013).
43. Deng B, Lin Y, Wang C, Li F, Wang Z, Zhang H, Li X-F, Le X C. Aptamer binding assays for proteins: The thrombin example—A review. Analytica Chimica Acta 837, 1-15 (2014).
44. Kelly J A, Feigon J, Yeates T O. Reconciliation of the X-ray and NMR structures of the thrombin-binding aptamer d(GGTTGGTGTGGTTGG). Journal of Molecular Biology 256, 417-422 (1996).
45. Wu X L, Xu L G, Liu L Q, Ma W, Yin H H, Kuang H, Wang L B, Xu C L, Kotov N A. Unexpected Chirality of Nanoparticle Dimers and Ultrasensitive Chiroplasmonic Bioanalysis. Journal of the American Chemical Society 135, 18629-18636 (2013).
46. Chen W, Bian A, Agarwal A, Liu L Q, Shen H B, Wang L B, Xu C L, Kotov N A. Nanoparticle Superstructures Made by Polymerase Chain Reaction: Collective Interactions of Nanoparticles and a New Principle for Chiral Materials. Nano letters 9, 2153-2159 (2009).
47. Dave V P, Ngo T A, Pernestig A-K, Tilevik D, Kant K, Nguyen T, et al. MicroRNA amplification and detection technologies: opportunities and challenges for point of care diagnostics. Laboratory Investigation. 99, 452-69 (2019).
48. Mitchell P S, Parkin R K, Kroh E M, Fritz B R, Wyman S K, Pogosova-Agadjanyan E L, et al. Circulating microRNAs as stable blood-based markers for cancer detection. Proceedings of the National Academy of Sciences of the United States of America. 105, 10513-8 (2008).
49. Ang Y S, Yung L-Y L. Rapid and Label-Free Single-Nucleotide Discrimination via an Integrative Nanoparticle-Nanopore Approach. ACS nano 6, 8815-8823 (2012).

1. A method of detecting one or more analytes in a target sample, the method comprising:
a. providing a nanoparticle dimer adapted to bind the analyte;
b. causing the dimer to pass through a nanopore by voltage-driven translocation;
c. observing changes in the translocation current; and
d. comparing the translocation current profile of the target sample to the translocation current profile of a control sample;
wherein a change in the translocation current profile of the target sample versus the control sample indicates the presence of the analyte in the target sample.

2. The method of claim 1, wherein the dimer comprises two nanoparticles linked by one or more nucleic acids, wherein at least one of the nucleic acids includes an aptamer specific for the analyte.

3. The method of claim 2, wherein the nanoparticles are linked by partially complementary nucleic acids.

4. The method of claim 1, wherein the dimer comprises two nanoparticles, each of which is attached to a nucleic acid, and each of the nucleic acids includes part of an aptamer specific for the analyte, such that in the presence of the analyte an aptamer is formed and thereby a dimer is formed.

5. The method of claim 1, wherein the analyte is a protein.

6. The method of claim 1, wherein the dimer comprises two nanoparticles, each of which is modified with a single stranded DNA (ssDNA), wherein one ssDNA includes a sequence which is complementary to the sequence of one end of the analyte, and the other ssDNA includes a sequence which is complementary to the sequence of the other end of the analyte, such that in the presence of the analyte a dimer is formed.

7. The method of claim 6, wherein the analyte is an miRNA.

8. The method of claim 1, wherein the dimer comprises two nanoparticles, each of which is conjugated to an antibody specific to a different epitope on the analyte, such that in the presence of the analyte a dimer is formed.

9. The method of claim 8, wherein the analyte is an antigen.

10. The method of claim 1, wherein the nanoparticles in the dimer are of substantially the same diameter.

11. The method of claim 1, wherein the nanoparticles in the dimer are of different diameters.

12. The method of claim 1, wherein the nanopore is the tip of a nanopipette.

13. A method of detecting one or more analytes in a target sample, the method comprising:
a. providing a nanoparticle adapted to bind the analyte;
b. providing a carrier nucleic acid molecule with at least one single-stranded region;
c. contacting the carrier nucleic acid molecule and nanoparticle with the target sample, forming a carrier nucleic acid/analyte/nanoparticle complex;
d. causing the carrier nucleic acid/analyte/nanoparticle complex to pass through a biological nanopore by voltage-driven translocation;
e. observing changes in the translocation current; and
f. comparing the translocation current profile of the target sample to the translocation current profile of a control sample;
wherein a change in the translocation current profile of the target sample versus the control sample indicates the presence of the analyte in the target sample.

14. The method of claim 13, wherein the carrier nucleic acid molecule includes an aptamer specific for the analyte and the nanoparticle is conjugated to an antibody specific to a different epitope on the analyte from that to which the aptamer binds.

15. The method of claim 13, wherein the analyte is a protein.

16. The method of claim 13, wherein the carrier nucleic acid molecule is a ssDNA which includes a sequence that is complementary to the sequence of one end of the analyte and the nanoparticle is modified with a ssDNA which includes a sequence that is complementary to the sequence of the other end of the analyte.

17. The method of claim 16, wherein the analyte is an miRNA.

18. The method of claim 13, wherein the biological nanopore is alpha-hemolysin.

19. The method of claim 13, wherein the nanoparticle is a gold nanoparticle (AuNP).

20. The method of claim 13, wherein the target sample is a biological sample selected from blood, serum, lymph, sputum, urine, faeces, semen, sweat, tears, amniotic fluid, cerebrospinal fluid (CSF) and wound exudate.
